(19) United States
(12) Patent Application Publication
(10) Pub. No.: US 2026/0211322 A1
(43) Pub. Date: Jul. 23, 2026

(54) COMPOSITION FOR CROSSLINKING, AND METHOD FOR PATTERN FORMATION, PATTERN FILM, AND ELECTRONIC DEVICE USING THE SAME

(71) Applicants: UNIST (ULSAN NATIONAL INSTITUTE OF SCIENCE AND TECHNOLOGY), Ulsan (KR); UIF (UNIVERSITY INDUSTRY FOUNDATION), YONSEI UNIVERSITY, Seoul (KR)

(72) Inventors: BongSoo KIM, Ulsan (KR); Hyobin HAM, Ulsan (KR); Jeong Ho CHO, Seoul (KR); In Cheol KWAK, Seoul (KR)

(73) Assignees: UNIST (ULSAN NATIONAL INSTITUTE OF SCIENCE AND TECHNOLOGY), Ulsan (KR); UIF (UNIVERSITY INDUSTRY FOUNDATION), YONSEI UNIVERSITY, Seoul (KR)

(21) Appl. No.: 19/456,318

(22) Filed: Jan. 22, 2026

(30) Foreign Application Priority Data
Jan. 23, 2025 (KR) 10-2025-0010682
Jan. 20, 2026 (KR) 10-2026-0011291

(51) Int. Cl. G03F 7/004; C07C 249/00; H10K 85/10; H10P 76/20

(52) U.S. Cl. CPC G03F 7/0045; C07C 249/00; H10K 85/113; H10K 85/151; H10P 76/204

(57) ABSTRACT

Provided are a crosslinking composition, and a pattern formation method, a pattern film, and an electronic device each using the same.

### CROSS-REFERENCE TO RELATED APPLICATION

This application is based on and claims priority under 35 U.S.C. § 119 to Korean Patent Application Nos. 10-2025-0010682, filed on Jan. 23, 2025, and 10-2026-0011291, filed on Jan. 20, 2026 in the Ministry of Intellectual Property, Republic of Korea, the disclosures of which are incorporated by reference herein in their entirety.

### BACKGROUND

### 1. Field

The disclosure relates to a crosslinking composition, and a pattern formation method, a pattern film, and an electronic device each using the same.

This research was conducted with the support of Samsung Science & Technology Foundation (Project Number: SRFC-MA1901-51).

### 2. Description of the Related Art

As compared to methods of manufacturing electronic devices through vacuum-based processes according to a related art, methods of manufacturing electronic devices through solution-based processes have an advantage of significantly reducing process costs. However, technology for patterning materials used in manufacturing electronic devices into respective material layers through a solution process generally requires the use of a photosensitive resin and an etching process, resulting in complicated processes and potential damage to a material to be patterned. In particular, in a patterning process of a material layer through a solution process, damage to a lower layer occurs when an upper layer is formed, resulting in a deterioration in the characteristics of electrical devices. Such problems may occur not only in single devices but also in the manufacturing of large-area arrays of electronic devices or circuits.

### SUMMARY

Provided are a crosslinking composition having high fidelity and excellent crosslinking efficiency, and a pattern formation method, a pattern film, and an electronic device each using the same.

Additional aspects will be set forth in part in the description which follows and, in part, will be apparent from the description, or may be learned by practice of the presented embodiments of the disclosure.

According to an aspect of the disclosure, a crosslinking composition includes a crosslinking compound (i.e, crosslinker) including a diazo group, and an organic semiconductor material, a polymer material, a metal material, an insulating material, or any combination thereof.

According to another aspect of the disclosure, a pattern formation method includes forming a lower film by applying a crosslinking composition onto a substrate, and patterning the lower film, wherein the crosslinking composition includes a crosslinking compound including a diazo group, and an organic semiconductor material, a polymer material, a metal material, an insulating material, or any combination thereof.

According to another aspect of the disclosure, a pattern film is formed by using the crosslinking composition.

According to another aspect of the disclosure, an electronic device includes a pattern formed by using the crosslinking composition.

According to another aspect of the disclosure, a color filter includes a pattern formed by using the crosslinking composition.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1 to 3 are cross-sectional views illustrating a pattern (pattern film) formation method using a crosslinking composition, according to an embodiment;

FIGS. 4A and 4B are views illustrating a structure of an organic thin-film transistor according to an embodiment;

FIG. 5 shows height profiles of patterns formed by using crosslinking compositions according to Examples and Comparative Examples of the disclosure;

FIGS. 6A to 6D are an image and graphs showing height profiles, width profiles, and taper angles of patterns formed by using crosslinking compositions according to Examples and Comparative Examples of the disclosure, wherein the image is captured by using an atomic force microscope (AFM);

FIG. 7A shows pattern height retention according to crosslinking compound fraction for crosslinking compounds according to Examples and Comparative Examples of the disclosure;

FIG. 7B shows a crosslinking compound fraction for achieving a pattern height retention of 0.8 for crosslinking compounds according to Examples and Comparative Examples of the disclosure;

FIGS. 8A to 8C illustrate results of evaluating operational stability and environmental stability of organic thin-film transistors including patterns formed by using crosslinking compositions according to Example and Comparative Examples of the disclosure; and

FIG. 9 is a graph showing a transfer curve according to a Device Manufacturing Example and a Comparative Device Manufacturing Example of the disclosure.

### DETAILED DESCRIPTION

Reference will now be made in detail to embodiments, examples of which are illustrated in the accompanying drawings, wherein like reference numerals refer to like elements throughout. In this regard, the embodiments may have different forms and should not be construed as being limited to the descriptions set forth herein. Accordingly, the embodiments are merely described below, by referring to the figures, to explain aspects of the description.

Hereinafter, the disclosure will be described in more detail.

In the present specification, it will be understood that the terms such as “including,” “comprising,” and “having” specify the presence of stated features or components, but do not preclude the presence or addition of one or more other features or components.

In the present specification, it will be understood that when a component such as a layer or a film is referred to as being “on” another component, the component may be directly on the other component or intervening components may be present thereon.

### [Crosslinking Composition]

According to an embodiment of the disclosure, a crosslinking composition may include a crosslinking compound including a diazo group.

According to another embodiment, a crosslinking composition may include a crosslinking compound including a diazo group, and an organic semiconductor material, a polymer material, a metal material, an insulating material, or any combination thereof.

The crosslinking composition may include the crosslinking compound including the diazo group, and as will be described below, since the crosslinking compound according to the disclosure including the diazo group may be a low-temperature-activated crosslinking compound, a crosslinking reaction may occur even without a high-temperature heat treatment (for example, at a temperature exceeding 140° C.). Accordingly, a phenomenon, in which a pattern is damaged due to high temperature in a solution process to deteriorate fidelity characteristics, electrical characteristics, or the like, or change or degrade the morphology of a formed pattern thin film, may be prevented. In addition, since the crosslinking compound according to the disclosure has excellent miscibility and compatibility with organic semiconductor materials (that is, host materials) and other materials, a pattern formed by using the crosslinking composition according to the disclosure may have high fidelity and achieve excellent crosslinking efficiency.

According to an embodiment, the organic semiconductor material may be a host material.

According to an embodiment, the organic semiconductor material may include a conjugated polymer, a conjugated small molecule, a poly(diketopyrrolopyrrole) (PDPP)-based organic semiconductor material, a poly(naphthalene diimide) (PNDI)-based organic semiconductor material, or any combination thereof.

According to an embodiment, the organic semiconductor material may be a p-type organic semiconductor material, an n-type organic semiconductor material, or any combination thereof. For example, the PDPP-based organic semiconductor material may be a p-type organic semiconductor material, and the PNDI-based organic semiconductor material may be an n-type organic semiconductor material.

According to an embodiment, the polymer material may be any polymer material including a C—H bond. For example, the polymer material may be a vinyl-based polymer material. For example, the polymer material may be any polymer material formed through condensation polymerization.

According to an embodiment, the polymeric material may include polymethyl methacrylate (PMMA), polystyrene (PS), poly(vinylidene fluoride-co-hexafluoropropylene) (PVDF-HFP), or any combination thereof.

According to an embodiment, the metal material may include metal nanoparticles, quantum dot nanoparticles, or any combination thereof. For example, the metal nanoparticles may be AgNPs.

According to an embodiment, the crosslinking composition may have a three-dimensional structure.

According to an embodiment, the crosslinking compound may be a low-temperature-activated crosslinking compound.

According to an embodiment, the low-temperature-activated crosslinking compound may be thermally activated at a temperature of about 80° C. to about 130° C. to generate an intermediate. For example, the low-temperature-activated crosslinking compound may be thermally activated at a temperature of about 80° C. to about 120° C. to generate an intermediate.

According to an embodiment, the low-temperature-activated crosslinking compound may be activated by ultraviolet light. For example, the low-temperature-activated crosslinking compound may be activated by ultraviolet light having a wavelength of about 200 nm to about 380 nm, about 300 nm to about 380 nm, or about 365 nm.

According to an embodiment, as illustrated below, the diazo group of the crosslinking compound may induce carbene-mediated crosslinking by annealing.

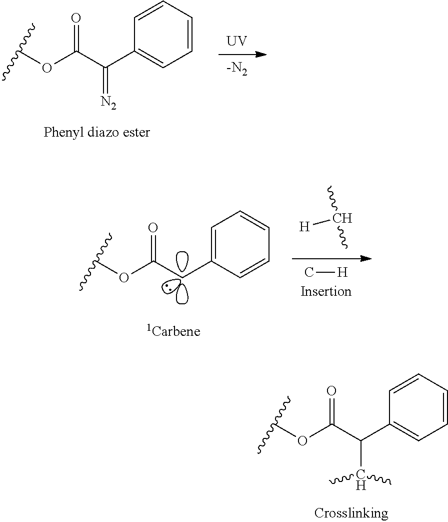

According to an embodiment, the crosslinking compound may be a compound represented by Formula 1 below:

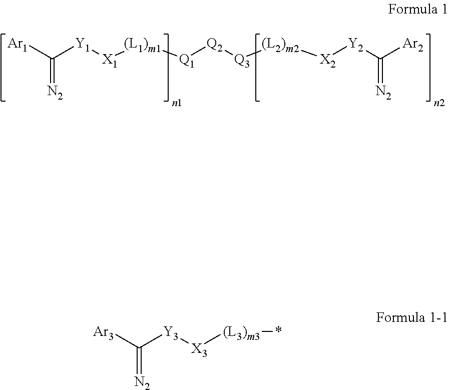

wherein, in Formulas 1 and 1-1,
L₁ to L₃ are each independently a single bond or a C₁-C₃₀ alkylene group unsubstituted or substituted with at least one R₁,
m1 to m3 are each independently 0, 1, 2, 3, 4, 5, or 6,
Ar₁ to Ar₃ are each independently a C₅-C₆₀ carbocyclic group unsubstituted or substituted with at least one R₁ or a C₁-C₆₀ heterocyclic group unsubstituted or substituted with at least one R₁,
n1 and n2 are each independently 0, 1, 2, 3, 4, 5, 6, 7, 8, 9, or 10,
the sum of n1 and n2 is greater than or equal to 2,
Q₁ to Q₃ are each independently a single bond, O, S, C, C(R₂), C(R₂)(R₃), a C₁-C₃₀ alkylene group unsubstituted or substituted with at least one R₁, or any combination thereof,
   X₁ to X₃ are each independently O, S, Se, N(R₄), or C(R₄)(R₅),
Y₁ to Y₃ are each independently *—C(═O)—*′, *—C(═N(R₆))—*′, *—O—*′, *—S—*′, *—Se—*′, *—N(R₆)—*′, or *—C(R₆)(R₇)—*′,
R₁ to R₇ are each independently a group represented by Formula 1-1, hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, a C₁-C₃₀ alkylthio group, a C₅-C₆₀ carbocyclic group, a C₁-C₆₀ heterocyclic group, or —Si(Q₁₁)(Q₁₂)(Q₁₃),
Q₁₁ to Q₁₃ are each independently hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group, and
and *′ is a binding site with an adjacent atom.

According to an embodiment, L₁ and L₂ may each independently be a single bond, a methylene group, or an ethylene group.

According to an embodiment, m1 and m2 may each independently be 1 or 2.

According to an embodiment, Ar₁ and Ar₂ may each independently be a substituted or unsubstituted phenyl group or benzene group.

According to an embodiment, Ar₃ may be a substituted or unsubstituted phenyl group or benzene group.

According to an embodiment, the sum of n1 and n2 may be 4 to 6.

According to an embodiment, Formula 1-1 may be a group represented by Formula 1-2 below:

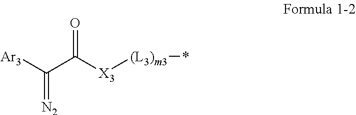

wherein, in Formula 1-2, L₃, m3, X₃, and Ar₃ are as defined herein.

According to an embodiment, the crosslinking compound may be a compound represented by Formula 2 below:

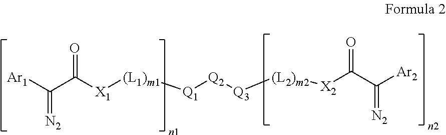

wherein, in Formula 2, L₁, L₂, m1, m2, n1, n2, X₁, X₂, Ar₁, Ar₂, and Q₁ to Q₃ are as defined herein.

According to an embodiment, the crosslinking compound may be a compound represented by Formula 3 below:

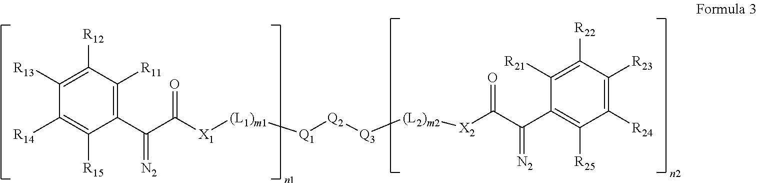

wherein, in Formula 3,
L₁, L₂, m1, m2, n1, n2, X₁, X₂, and Q₁ to Q₃ are as defined herein, and
R₁₁ to R₁₅ and R₂₁ to R₂₅ are each independently defined as for R₁.

According to an embodiment, the crosslinking compound may be at least one selected from Compounds 1 to 7 below:

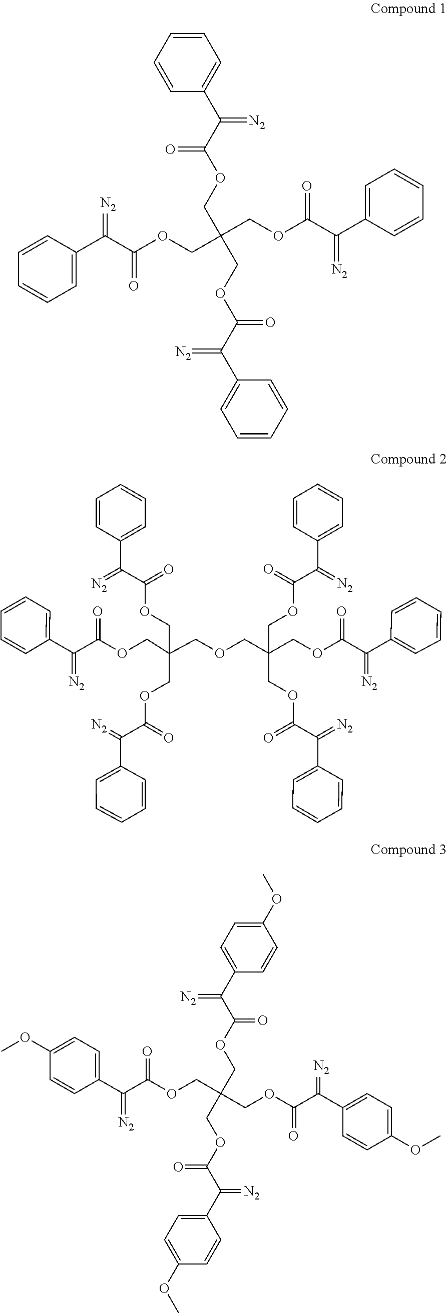

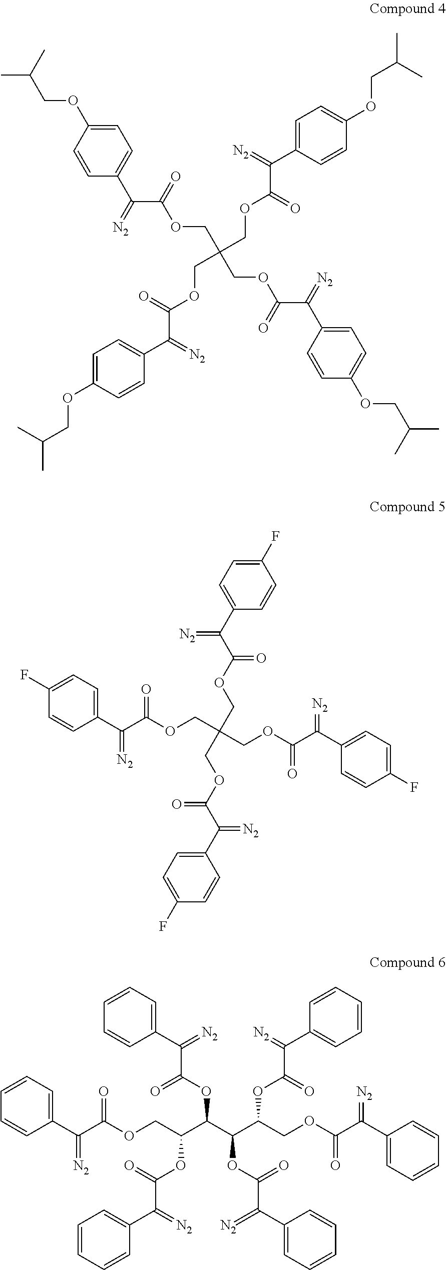

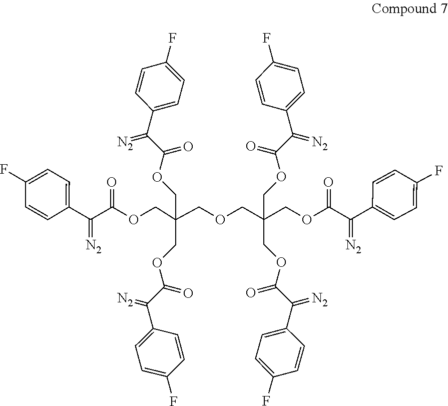

According to an embodiment, the organic semiconductor material may be at least one selected from compounds PDPP-1 and PNDI-1 below:

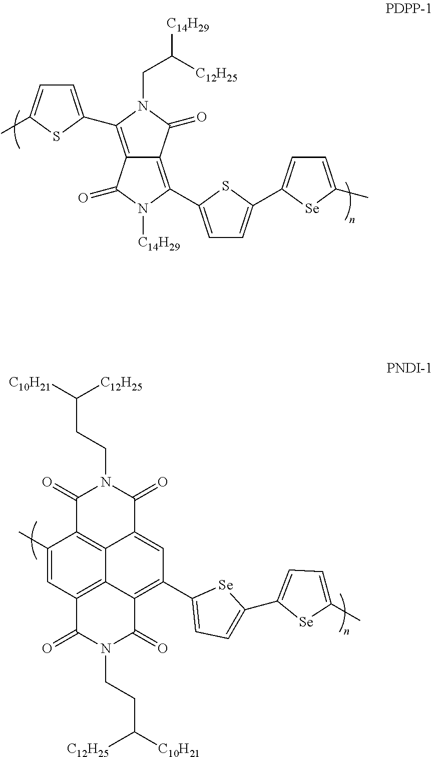

According to an embodiment, the crosslinking compound may form a bond with an organic semiconductor material, a polymer material, a metal material, an insulating material, or any combination thereof.

According to an embodiment, the crosslinking composition may further include a solvent.

According to an embodiment, the solvent may be an organic solvent. For example, the solvent may include chloroform, chlorobenzene, o-dichlorobenzene, 1,2,4-trichlorobenzene, toluene, xylene, tetralene, cyclohexylbenzene, octadecane, 1-octadecene (ODE), trioctylamine (TOA), trioctylphosphine (TOP), oleylamine, or any combination thereof.

According to an embodiment, the crosslinking composition may be used in a solution process.

### [Pattern Formation Method]

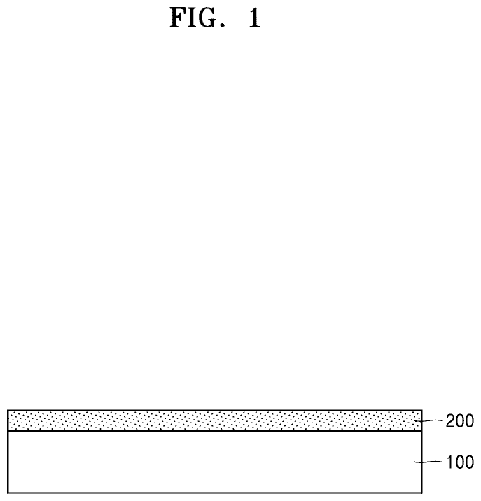

FIGS. 1 to 3 are cross-sectional views illustrating a pattern (pattern film) formation method using a crosslinking composition, according to an embodiment of the disclosure.

Referring to FIG. 1, a substrate 100 may be provided. A lower film 200 may be formed on the substrate 100. The lower film 200 may be formed through various coating methods that may be selected based on common technical knowledge. For example, the lower film 200 may be formed through spin coating, slot die coating, or bar coating. The lower film 200 may be provided as a single film or a plurality of stacked films. Although not shown, layers may be further provided between the substrate 100 and the lower film 200. The lower film 200 may be an etching target film.

The lower film 200 may include a crosslinking composition.

Accordingly, the lower film 200 may include the crosslinking compound, and an organic semiconductor material, a polymer material, a metal material, an insulating material, or any combination thereof.

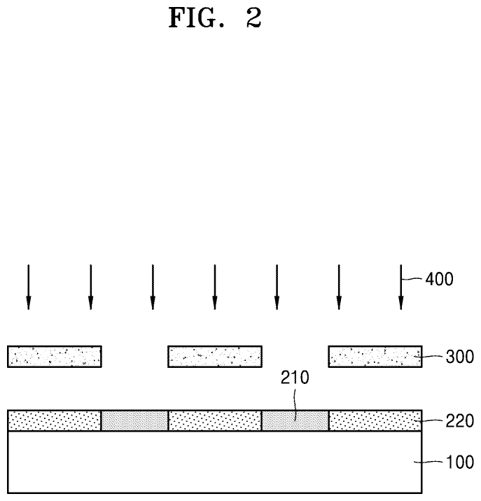

Referring to FIG. 2, a photomask 300 may be disposed on the lower film 200. The photomask 300 may expose a portion of the lower film 200. Light 400 may be irradiated onto the photomask 300. The lower film 200 may be exposed to the light 400. The light 400 may be an electron beam or ultraviolet light. The light 400 may be directly irradiated onto a first portion 210 of the lower film 200 exposed by the photomask 300. By the light 400, a crosslinking composition within the first portion 210 of the lower film 200 may undergo a crosslinking reaction to be cured.

Specifically, by the light 400, some of chemical bonds of a crosslinking compound in the crosslinking composition may be broken, thereby forming a compound including a neutral element having unshared electrons. For example, N₂ may be removed from a crosslinking compound, and a compound including neutral carbon (for example, carbene) including unshared electrons may be formed.

A second portion 220 of the lower film 200 that is not exposed by the photomask 300 may not be exposed to the light 400. That is, a crosslinking composition within the second portion 220 of the lower film 200 may not be cured.

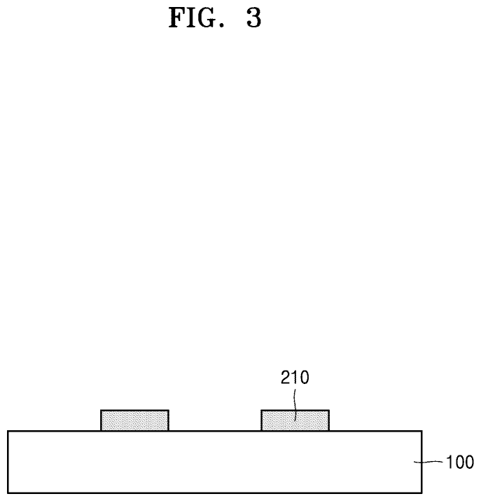

Referring to FIG. 3, the photomask 300 may be removed. The second portion 220 of the lower film 200 may be removed by using a solution, and the first portion 210 of the lower film 200 may remain to form a lower pattern. Hereinafter, the first portion 210 of the lower film 200 may be referred to as a lower pattern.

A crosslinking composition according to an embodiment of the disclosure may be used for forming a pattern or manufacturing an electronic device. For example, the crosslinking composition may be used in a patterning process of manufacturing an electronic device. According to the disclosure, a photoresist film may not be required for forming a pattern or manufacturing an electronic device. Therefore, a manufacturing process may be simplified, and damage to components of the electronic device may be simultaneously prevented. In addition, an electronic device manufactured by using a crosslinking composition may have improved electrical characteristics and stability.

Accordingly, referring to the pattern formation method, the pattern formation method according to the disclosure may include forming a lower film by applying a crosslinking composition onto a substrate, and patterning the lower film, wherein the crosslinking composition may include a crosslinking compound including a diazo group, and an organic semiconductor material, a polymer material, a metal material, an insulating material, or any combination thereof.

According to an embodiment, the patterning of the lower film may include arranging a photomask on the lower film, irradiating light onto the photomask, and removing a portion of the lower film.

The pattern formation method according to the disclosure may use a crosslinking composition according to an embodiment of the disclosure, and contents of the crosslinking composition described above may all be referenced to in the pattern formation method.

In the pattern formation method using a crosslinking composition, the crosslinking composition may be crosslinked by a crosslinking compound including a diazo group. Since the crosslinking compound may be a low-temperature-activated crosslinking compound, a crosslinking reaction may occur even without a high-temperature heat treatment (for example, at a temperature exceeding 140° C.). Accordingly, a phenomenon, in which a pattern is damaged due to high temperature in a solution process to deteriorate fidelity characteristics, electrical characteristics, or the like, or change or degrade the morphology of a formed pattern thin film, may be prevented. In addition, since the crosslinking compound according to the disclosure has excellent miscibility and compatibility with organic semiconductor materials (that is, host materials) and other materials, a pattern formed by using the crosslinking composition according to the disclosure may have high fidelity and achieve excellent crosslinking efficiency.

### [Pattern Film]

According to another aspect of the disclosure, provided is a pattern film formed by using the crosslinking composition.

According to an embodiment, the pattern film may be prepared through the pattern formation method described above.

According to an embodiment, the pattern film may include a substrate, and a pattern formed on the substrate. In addition, the substrate may be used interchangeably with the term “base material.”

According to an embodiment, the pattern film may include the substrate, and the pattern formed on the substrate, and the pattern may include a single thin film or a multilayer thin film.

In another embodiment, the pattern may consist of a single-layer thin film.

According to another embodiment, the pattern may include or consist of a thin film with a multilayer structure in which a plurality of single-layer thin films are stacked.

According to an embodiment, the pattern including the thin film with a multilayer structure may include a thin film formed according to the pattern formation method according to the disclosure and/or a thin film that may be formed according to common technology knowledge.

A pattern film prepared through the pattern formation method using a crosslinking composition may be crosslinked by a crosslinking compound including a diazo group. Since the crosslinking compound may be a low-temperature-activated crosslinking compound, a crosslinking reaction may occur even without a high-temperature heat treatment (for example, at a temperature exceeding 140° C.). Accordingly, a phenomenon, in which a pattern is damaged due to high temperature in a solution process to deteriorate fidelity characteristics, electrical characteristics, or the like, or change or degrade the morphology of a formed pattern thin film, may be prevented. In addition, since the crosslinking compound according to the disclosure has excellent miscibility and compatibility with organic semiconductor materials (that is, host materials) and other materials, a pattern formed by using the crosslinking composition according to the disclosure may have high fidelity and achieve excellent crosslinking efficiency.

According to an embodiment, the substrate or a base material may be selected in consideration of mechanical strength, thermal stability, surface smoothness, ease of handling, water resistance, and the like. For example, a silicon wafer or a glass substrate may be used, or a plastic film of polyethersulfone, polyacrylate, polyetherimide, polyimide, polyethylene naphthalate, or polyethylene terephthalate, or an organic substrate coated with any of these plastic films may be used.

According to an embodiment, the base material may have a single-layer structure or a multilayer structure.

For example, the base material may be a single layer including a resin. For another example, the base material may have a multilayer structure including two or more layers which respectively include different types of resins. For another example, the base material may have a multilayer structure including a resin-containing layer and a functional layer, and the functional layer may be, for example, an adhesive layer, an anti-corrosion layer, an anti-reflective layer, a hard coating layer, or a combination thereof.

According to an embodiment, the pattern may have a thickness of about 1 nm to about 5 μm. For example, the pattern may have a thickness of about 5 nm to about 1 μm, about 10 nm to about 500 nm, or about 15 nm to about 250 nm.

### [Electronic Device]

According to another aspect of the disclosure, provided is an electronic device including the pattern formed by using the crosslinking composition.

According to another aspect of the disclosure, provided is an electronic device including the pattern film formed by using the crosslinking composition.

According to an embodiment, the electronic device may be an organic thin-film transistor (OTFT), a logic electronic device, an organic light-emitting diode (OLED), a quantum dot light-emitting diode (QD-LED), an electrochromic (EC) device, a solar cell, a photodiode, or an image sensor.

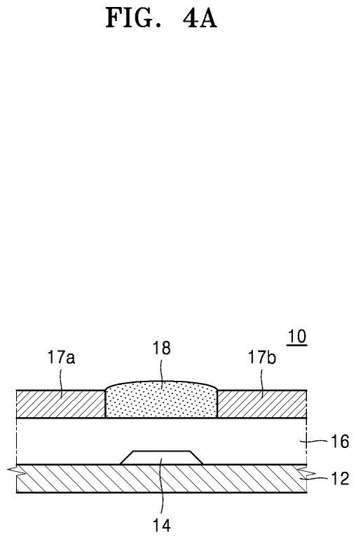

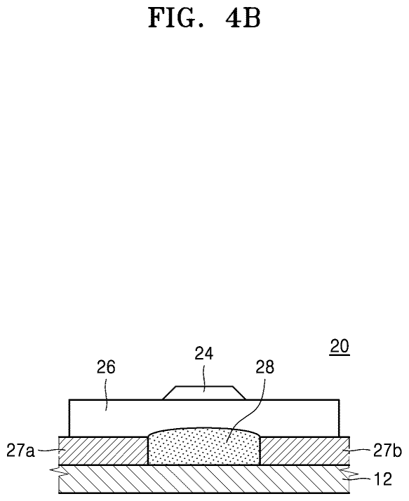

FIGS. 4A and 4B are schematic cross-sectional views of a transistor according to an embodiment of the disclosure.

Referring to FIG. 4A, in a transistor 10, a gate electrode 14 is formed on a substrate 12, and an insulating layer 16 covering the gate electrode 14 is formed. A source electrode 17a and a drain electrode 17b which define a channel region may be formed on the insulating layer 16, an active layer 18 may be formed in the channel region, and the active layer 18 may include a pattern film formed by using the crosslinking composition.

Referring to FIG. 4B, in a transistor 20, a source electrode 27a and a drain electrode 27b which define a channel region are formed on a substrate 22, an active layer 28 is formed in the channel region, and the active layer 28 includes a pattern film formed by using the crosslinking composition. An insulating layer 26 is formed to cover the source electrode 27a, the drain electrode 27b, and the active layer 28, and a gate electrode 24 is formed on the insulating layer 26.

The substrates 12 and 22 may include an inorganic material, an organic material, or a composite of an inorganic material and an organic material. Examples of the organic material may include plastic such as polyethylenenaphthalate (PEN), polyethyleneterephthalate (PET), polycarbonate, polyvinyl alcohol, polyacrylate, polyimide, polynorbornene, or polyethersulfone (PES), and examples of the inorganic material may include glass or metal.

In addition, as the gate electrodes 14 and 24, the source electrodes 17a and 27a, and the drain electrodes 17b and 27b, a commonly used metal may be used. Specifically, gold (Au), silver (Ag), aluminum (Al), nickel (Ni), indium tin oxide (ITO), or the like may be used, but one or more embodiments not limited thereto.

As the insulating layers 16 and 26, a commonly used high dielectric constant insulator may be used. Specifically, a ferroelectric insulator such as Ba_{0.33}Sr_{0.66}TiO₃ (barium strontium titanate (BST)), Al₂O₃, Ta₂O₅, La₂O₅, Y₂O₃, or TiO₂, an inorganic insulator such as PbZr_{0.33}Ti_{0.66}O₃ (PZT), Bi₄Ti₃O₁₂, BaMgF₄, SrBi₂(TaNb)₂O₉, Ba(ZrTi)O₃(BZT), BaTiO₃, SrTiO₃, SiO₂, SiNₓ (x is determined by a valence of Si), or AION, or an organic insulator such as polyimide, benzocyclobutene (BCB), parylene, polyacrylate, polyvinylalcohol, polyvinylphenol, or polyvinylphenol may be used, but one or more embodiments are not limited thereto.

### [Color Filter]

According to another aspect of the disclosure, provided is a color filter including the pattern.

According to another aspect of the disclosure, provided is a color filter including the pattern film.

For example, in a display device, light emitted from a backlight source may pass through the color filter to form colors. Accordingly, the color filter of the disclosure may include the pattern formed by using the crosslinking composition provided herein, and the color filter may be used in the display device or the like to form colors.

### [Definition of Substituent]

In the present specification, a C₅-C₆₀ carbocyclic group refers to a monocyclic or polycyclic group having 5 to 60 carbon atoms and including only carbon as a ring-forming atom. The C₅-C₆₀ carbocyclic group may be an aromatic carbocyclic group or a non-aromatic carbocyclic group. The C₅-C₆₀ carbocyclic group may be a ring such as benzene, a monovalent group such as a phenyl group, or a divalent group such as a phenylene group. Alternatively, according to the number of substituents connected to the C₅-C₆₀ carbocyclic group, the C₅-C₆₀ carbocyclic group may be variously modified into a trivalent group, a tetravalent group, or the like.

In the present specification, a C₁-C₆₀ heterocyclic group refers to a group having the same structure as the C₅-C₆₀ carbocyclic group, except that the group includes, as a ring-forming atom, at least one heteroatom selected from N, O, Si, P, and S in addition to carbon (the number of carbon atoms may be 1 to 60).

In the present specification, the C₁-C₃₀ alkyl group refers to a linear or branched aliphatic hydrocarbon group having 1 to 30 carbon atoms, and specific examples thereof include a methyl group, an ethyl group, a propyl group, an isobutyl group, a sec-butyl group, a ter-butyl group, a pentyl group, an iso-amyl group, a hexyl group, a heptyl group, an n-octyl group, a 2-ethylhexyl group, and the like.

In the present specification, a C₂-C₃₀ alkenyl group refers to a hydrocarbon group including one or more carbon-carbon double bonds in the middle or terminus of the C₂-C₃₀ alkyl group, and specific examples thereof include an ethenyl group, a propenyl group, a butenyl group, and the like.

In the present specification, the C₂-C₃₀ alkynyl group refers to a hydrocarbon group including one or more carbon-carbon triple bonds in the middle or terminus of the C₂-C₃₀ alkyl group, and specific examples thereof include an ethynyl group, a propynyl group, and the like.

In the present specification, a C₁-C₃₀ alkoxy group refers to a monovalent group having a formula of —OA₁₀₁ (A₁₀₁ is the C₁-C₃₀ alkyl group), and specific examples thereof include a methoxy group, an ethoxy group, an isopropyloxy group, and the like.

In the present specification, a C₁-C₃₀ alkylthio group refers to a monovalent group having a formula of —SA₁₀₁ (A₁₀₁ is the C₁-C₃₀ alkyl group), and specific examples thereof include a methylthio group, an ethylthio group, an isopropylthio group, and the like.

As used herein, unless otherwise defined, the symbols “* and *′” each denote a bonding site with an adjacent atom in a corresponding formula.

Hereinafter, the disclosure will be described in more detail by way of examples. These examples are for describing the disclosure in more detail, and the scope of the disclosure is not limited by the examples.

### EXAMPLES

### Synthesis Example 1: Preparation of Compounds 1 to 7

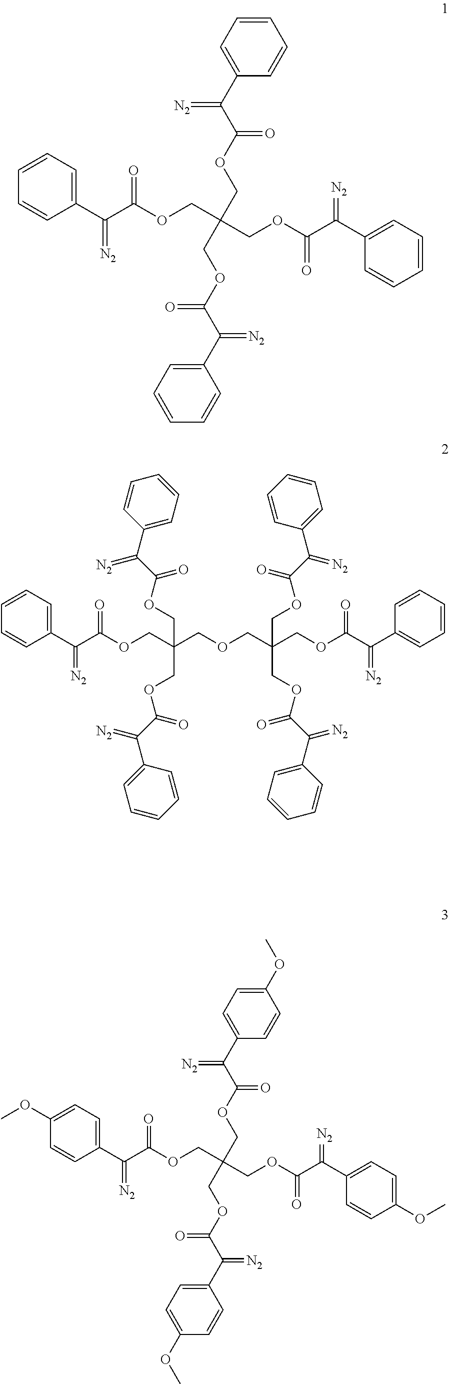

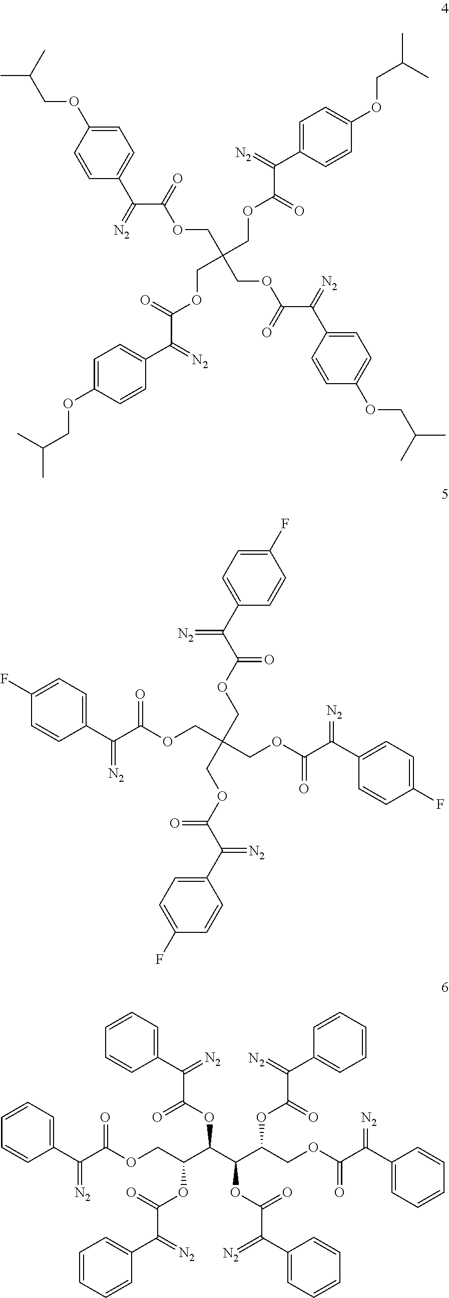

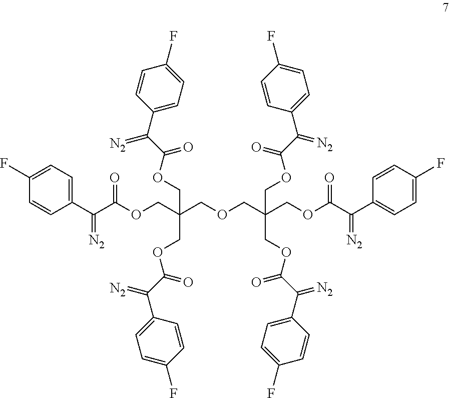

### (1) Preparation of Crosslinking Compound 1

A mixed solution of phenyl acetyl chloride (1,020 mg, 6.60 mmol) and pentaerythritol (150 mg, 1.10 mmol) was stirred at a temperature of 140° C. for 18 hours under an argon atmosphere. Then, the mixed solution was cooled to room temperature, and then extraction was performed by using dichloromethane (DCM) and distilled water. An organic layer was dried over MgSO₄, filtered, and then concentrated by using a rotary evaporator. A material was purified by silica gel column chromatography (eluent: ethyl acetate:hexane=1:4 volume ratio) to obtain a white solid (483 mg, 72%). ¹H-NMR (400 MHz, CDCl₃) δ: 7.29-7.15 (m, 20H), 3.87 (s, 8H), 3.51 (s, 8H).

Under an argon atmosphere, the white solid (400 mg, 0.66 mmol) obtained above and p-ABSA (789 mg, 3.28 mmol) were dissolved in anhydrous tetrahydrofuran (THF) (10 mL) and stirred for 10 minutes. 1,8-diazabicyclo[5.4.0]undec-7-ene (DBU) (670 mg, 4.40 mmol) was slowly added dropwise to a solution and then stirred at room temperature for 12 hours. Next, extraction was performed by using DCM and distilled water. An organic layer was dried over MgSO₄, filtered, and then concentrated by using a rotary evaporator. A material was purified by silica gel column chromatography (eluent: ethyl acetate:hexane=1:5 volume ratio). An obtained material was recrystallized by using chloroform and methanol to obtain crosslinking compound 1 as an orange solid (433 mg, 92%). ¹H-NMR (400 MHz, CDCl₃) δ: 7.43-7.34 (m, 16H), 7.21-7.16 (t, J=8.0 Hz, 4H), 4.41 (s, 8H).

### (2) Preparation of Crosslinking Compound 2

A mixed solution of phenyl acetyl chloride (2,193 mg, 14.18 mmol) and dipentaerythritol (500 mg, 1.97 mmol) was stirred at a temperature of 120° C. for 18 hours under an argon atmosphere. The mixed solution was cooled to room temperature, and then extraction was performed by using DCM and distilled water. An organic layer was dried over MgSO₄, filtered, and then concentrated by using a rotary evaporator. A material was purified by silica gel column chromatography (eluent:ethyl acetate:hexane=1:3 volume ratio) to obtain a viscous yellow liquid (1,506 mg, 80%). ¹H-NMR (400 MHz, CDCl₃) δ: 7.31-7.18 (m, 30H), 3.87 (s, 12H), 3.53 (s, 12H), 2.87 (s, 4H).

Under an argon atmosphere, the viscous yellow liquid (1,000 mg, 1.04 mmol) obtained above and p-ABSA (1,795 mg, 7.47 mmol) were dissolved in anhydrous acetonitrile (20 mL) and stirred for 10 minutes. DBU (1,580 mg, 10.38 mmol) was slowly added dropwise to a solution and then stirred at room temperature for 24 hours. Next, extraction was performed by using DCM and distilled water. An organic layer was dried over MgSO₄, filtered, and then concentrated by using a rotary evaporator, and then a material was purified by using silica gel column chromatography (eluent=DCM). An obtained material was recrystallized by using chloroform and methanol to obtain crosslinking compound 2 as an orange solid (577 mg, 49%). ¹H-NMR (400 MHz, CDCl₃) δ: 7.39-7.33 (m, 24H), 7.17-7.13 (t, 6H), 4.37 (s, 12H).

### (3) Preparation of Crosslinkinq Compound 3

A mixed solution of 4-methoxyphenyl acetyl chloride (1,605 mg, 11.02 mmol) and pentaerythritol (300 mg, 2.203 mmol) was stirred at a temperature of 120° C. for 12 hours under an argon atmosphere. The mixed solution was cooled to room temperature, and then extraction was performed by using DCM and distilled water. An organic layer was dried over MgSO₄, filtered, and then concentrated by using a rotary evaporator. A material was purified by silica gel column chromatography (eluent: ethyl acetate:hexane=1:4 volume ratio) to obtain a white solid (1,498 mg, 93%). ¹H-NMR (400 MHz, CDCl₃) δ: 7.09 (d, J=8 Hz, 8H), 6.82 (d, J=8 Hz, 8H), 3.91 (s, 8H), 3.77 (s, 12H), 3.46 (s, 8H).

Under an argon atmosphere, the white solid (300 mg, 0.412 mmol) obtained above and p-ABSA (611 mg, 2.470 mmol) were dissolved in anhydrous acetonitrile (7 mL) and stirred for 10 minutes. DBU (376 mg, 2.470 mmol) was slowly added dropwise to a solution and then stirred at room temperature for 24 hours. Next, extraction was performed by using DCM and distilled water. An organic layer was dried over MgSO₄, filtered, and then concentrated by using a rotary evaporator. A material was purified by silica gel column chromatography (eluent: ethyl acetate:DCM=1:20 volume ratio). An obtained material was recrystallized by using chloroform and methanol to obtain crosslinking compound 3 as an orange solid (433 mg, 92%). ¹H-NMR (400 MHz, CDCl₃) δ: 7.31 (d, J=8 Hz, 8H), 6.91 (d, J=8 Hz, 8H), 4.36 (s, 8H), 3.80 (s, 12H).

### (4) Preparation of Crosslinking Compound 4

A mixed solution of 4-methoxyphenyl acetyl chloride (1,605 mg, 11.02 mmol) and pentaerythritol (300 mg, 2.203 mmol) was stirred at a temperature of 120° C. for 12 hours under an argon atmosphere. The mixed solution was cooled to room temperature, and then extraction was performed by using DCM and distilled water. An organic layer was dried over MgSO₄, filtered, and then concentrated by using a rotary evaporator. A material was purified by silica gel column chromatography (eluent: ethyl acetate:hexane=1:4 volume ratio) to obtain a white solid (1,498 mg, 93%). ¹H-NMR (400 MHz, CDCl₃) δ: 7.09 (d, J=8 Hz, 8H), 6.82 (d, J=8 Hz, 8H), 3.91 (s, 8H), 3.77 (s, 12H), 3.46 (s, 8H).

The white solid (1,000 mg, 1.372 mmol) obtained above was dissolved in anhydrous DCM (40 mL) under an argon atmosphere and stirred at a temperature of −78° C. for 1 hour. Then, a BBr₃ solution (1 M in DCM, 6.2 mL, 6.174 mmol) was slowly added dropwise and then stirred at a temperature of 0° C. for 7 hours. After the reaction was terminated by slowly adding a saturated NaHCO₃ aqueous solution, extraction was performed with ethyl acetate. An organic layer was dried over MgSO₄, filtered, and then concentrated by using a rotary evaporator. An obtained material was precipitated with hexane to form a white solid (551 mg, 60%). ¹H-NMR (400 MHz, DMSO-d₆) δ: 8.33 (s, 4H), 6.98 (d, J=8 Hz, 8H), 6.67 (d, J=8 Hz, 8H), 3.94 (s, 8H), 3.46 (s, 8H)

Under an argon atmosphere, the white solid (100 mg, 0.149 mmol) obtained above, 1-bromo-2-methyl-propane (611 mg, 2.470 mmol), and K₂CO₃ (144 mg, 1.043 mmol) were dissolved in anhydrous N,N-dimethylformamide (DMF) (7 mL) and stirred at temperature of 110° C. for 24 h. A mixed solution was cooled to room temperature, and then extraction was performed by using DCM and distilled water. An organic layer was dried over MgSO₄, filtered, and then concentrated by using a rotary evaporator. A material was purified by silica gel column chromatography (eluent: ethyl acetate:hexane=1:3 volume ratio) to obtain a viscous liquid product (73 mg, 54%). ¹H-NMR (400 MHz, CDCl₃) δ: 7.07 (d, J=8 Hz, 8H), 6.81 (d, J=8 Hz, 8H), 3.93 (s, 8H), 3.67 (d, J=8 Hz, 8H), 3.45 (s, 12H), 2.09-2.02 (m, 4H), 1.01 (d, J=4 Hz, 24H).

Under an argon atmosphere, the liquid (73 mg, 0.081 mmol) obtained above and p-ABSA (74 mg, 0.486 mmol) were dissolved in anhydrous acetonitrile (5 mL) and stirred for 10 minutes. DBU (117 mg, 0.486 mmol) was slowly added dropwise to a solution and then stirred at room temperature for 24 hours. Next, extraction was performed by using DCM and distilled water. An organic layer was dried over MgSO₄, filtered, and then concentrated by using a rotary evaporator. A material was purified by silica gel column chromatography (eluent: ethyl acetate:hexane=1:5 volume ratio). An obtained material was recrystallized by using DCM and methanol to obtain crosslinking compound 4 as an orange solid (9 mg, 11%). ¹H-NMR (400 MHz, CDCl₃) δ: 7.29 (d, J=8 Hz, 8H), 6.90 (d, J=8 Hz, 8H), 4.34 (s, 8H), 3.70 (d, J=4 Hz, 8H), 2.10-2.03 (m, 4H), 1.02 (d, J=4 Hz, 24H).

### (5) Preparation of Crosslinking Compound 5

A mixed solution of 4-fluorophenyl acetyl chloride (1,267 mg, 7.345 mmol) and pentaerythritol (200 mg, 1.469 mmol) was stirred at a temperature of 140° C. for 18 hours. The mixed solution was cooled to room temperature, and then extraction was performed by using ethyl acetate and distilled water. An organic layer was dried over MgSO₄, filtered, and then concentrated by using a rotary evaporator. A material was purified by silica gel column chromatography (eluent: ethyl acetate:hexane=1:2 volume ratio) to obtain a white solid (806 mg, 81%). ¹H-NMR (400 MHz, CDCl₃) δ: 7.14 (dd, J=8 Hz, 4 Hz, 8H), 6.99 (t, J=8 Hz, 8H), 3.93 (s, 8H), 3.51 (s, 8H).

Under an argon atmosphere, the white solid (400 mg, 0.588 mmol) obtained above and p-ABSA (1019 mg, 4.114 mmol) were dissolved in anhydrous acetonitrile (10 mL) and stirred for 10 minutes. Then, DBU (626 mg, 2.470 mmol) was slowly added dropwise and then stirred at room temperature for 24 hours. Next, extraction was performed by using DCM and distilled water. An organic layer was dried over MgSO₄, filtered, and then concentrated by using a rotary evaporator. A material was purified by silica gel column chromatography (eluent=DCM). An obtained material was recrystallized by using chloroform and methanol to obtain crosslinking compound 5 as an orange solid (78 mg, 17%). ¹H-NMR (400 MHz, CDCl₃) δ: 7.38 (t, J=8 Hz, 8H), 7.08 (t, J=8 Hz, 8H), 4.38 (s, 8H).

### (6) Preparation of Crosslinking Compound 6

A mixed solution of phenyl acetyl chloride (7,636 mg, 49.40 mmol) and D-mannitol (1,000 mg, 5.489 mmol) was stirred at a temperature of 130° C. for 24 hours under an argon atmosphere. The mixed solution was cooled to room temperature, and then extraction was performed by using DCM and distilled water. An organic layer was dried over MgSO₄, filtered, and then concentrated by using a rotary evaporator. A material was purified by silica gel column chromatography (eluent: ethyl acetate:hexane=1:5 volume ratio) to obtain a pale yellow liquid (1,177 mg, 24%). ¹H-NMR (400 MHz, CDCl₃) δ: 7.32-7.19 (m, 30H), 5.46 (d, J=8 Hz, 2H), 5.11-5.07 (m, 2H), 4.20 (s, 1H), 4.19 (s, 1H), 3.88 (d, J=4 Hz, 1H), 3.85 (d, J=4 Hz, 1H), 3.60 (d, J=4 Hz, 4H), 3.54 (d, J=4 Hz, 4H), 3.50 (s, 4H).

Under an argon atmosphere, the pale yellow liquid (1,157 mg, 1.299 mmol) obtained above and p-ABSA (2,808 mg, 11.69 mmol) were dissolved in anhydrous acetonitrile (50 mL) and stirred for 10 minutes. Then, DBU (1,780 mg, 11.69 mmol) was slowly added dropwise and then stirred at room temperature for 24 hours. Next, extraction was performed by using DCM and distilled water. An organic layer was dried over MgSO₄, filtered, and then concentrated by using a rotary evaporator. A material was purified by silica gel column chromatography (eluent=DCM). An obtained material was recrystallized by using chloroform and methanol to obtain crosslinking compound 6 as a yellow solid (279 mg, 21%). ¹H-NMR (400 MHz, CDCl₃) δ: 7.40-7.14 (m, 30H), 5.81 (d, J=8 Hz, 2H), 5.49 (m, 2H), 4.70 (d, J=12 Hz, 2H), 4.35 (dd, J=12 Hz, 4 Hz, 2H).

### (7) Preparation of Crosslinking Compound 7

A mixed solution of 4-fluorophenyl acetyl chloride (1,010 mg, 5.852 mmol) and dipentaerythritol (250 mg, 0.836 mmol) was stirred at a temperature of 120° C. for 18 hours. The mixed solution was cooled to room temperature, and then extraction was performed by using DCM and distilled water. An organic layer was dried over MgSO₄, filtered, and then concentrated by using a rotary evaporator. A material was purified by silica gel column chromatography (eluent: ethyl acetate:hexane=1:2 volume ratio) to obtain a white solid (710 mg, 79%). ¹H-NMR (400 MHz, CDCl₃) δ: 7.17 (t, J=4 Hz, 4 Hz, 12H), 6.99 (t, J=4 Hz, 12H), 3.92 (s, 12H), 3.52 (s, 12H), 2.94 (s, 4H).

Under an argon atmosphere, the white solid (400 mg, 0.374 mmol) obtained above and p-ABSA (897 mg, 3.735 mmol) were dissolved in anhydrous acetonitrile (10 mL) and stirred for 10 minutes. Then, DBU (568 mg, 3.735 mmol) was slowly added dropwise and then stirred at room temperature for 24 hours. Next, extraction was performed by using DCM and distilled water. An organic layer was dried over MgSO₄, filtered, and then concentrated by using a rotary evaporator. A material was purified by silica gel column chromatography (eluent: chloroform:hexane=30:1 volume ratio). An obtained material was recrystallized by using chloroform and hexane to obtain crosslinking compound 7 as an orange solid (160 mg, 35%). ¹H-NMR (400 MHz, CDCl₃) δ: 7.34 (t, J=4 Hz, 12H), 7.03 (t, J=8 Hz, 12H), 4.34 (s, 12H), 3.46 (s, 4H).

### Synthesis Example 2: Preparation of Compounds PDPP-1 and PNDI-1

### Preparation of Compound PDPP-1

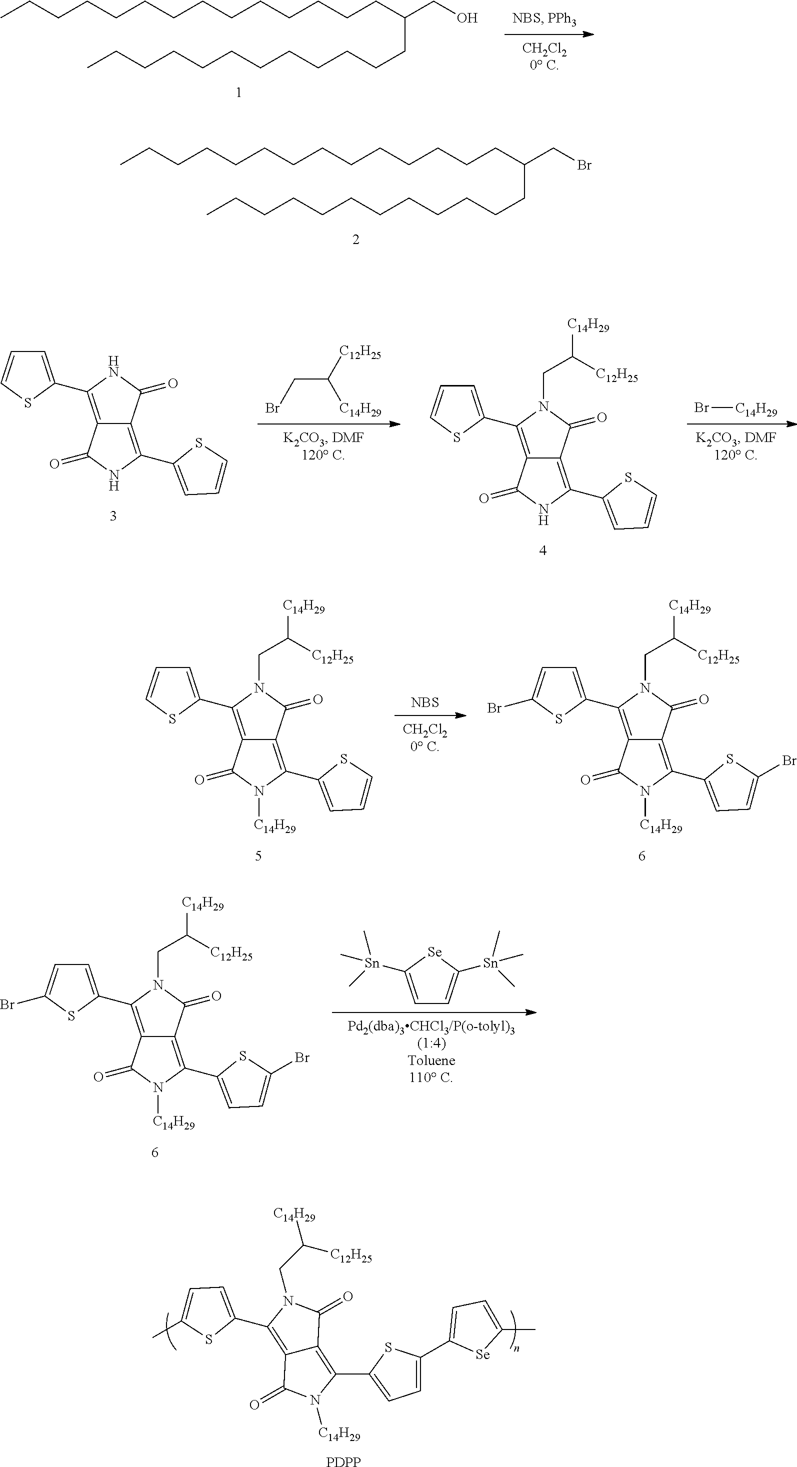

### 1) Synthesis of Intermediate 2

2-dodecylhexadecan-1-ol (1) (20.00 g, 48.68 mmol) and triphenylphosphine (19.15 g, 73.02 mmol) were dissolved in anhydrous DCM (117 mL). N-bromosuccinimide (13 g, 73.02 mmol) was added portionwise at a temperature of 0° C. Afterwards, a reaction solution was stirred at room temperature for 19 hours, and then the reaction was stopped by using a saturated Na₂S₂O₃ aqueous solution. An organic layer was extracted three times with DCM (150 mL) and dried over anhydrous MgSO₄. After a solvent was removed by using a rotary evaporator, a crude product was purified by silica gel column chromatography using n-hexane as an eluent. A final product was obtained in the form of colorless oil in a high yield of 99% (35.02 g).

¹H-NMR (400 MHz, CDCl₃): δ=3.45-3.44 (d, 2H), 1.61-1.53 (m, 1H), 1.41-1.20 (m, 40H), 0.90-0.86 (t, 6H).

### 2) Synthesis of Intermediate 4

3,6-di(thiophen-2-yl)-2,5-dihydropyrrolo[3,4-c]pyrrole-1,4-dione (3) (10.00 g, 33.29 mmol) and K₂CO₃ (5.98 g, 43.28 mmol) were added to anhydrous DMF (303 mL). A solution was stirred at a temperature of 120° C. for 1 hour. Thereafter, DH-Br (15.73 g, 33.29 mmol) was added to a reaction solution and stirred at a temperature of 120° C. for 15 hours. A reaction mixture was cooled to room temperature, a solvent was removed, and then deionized water (100 mL) and chloroform (100 mL) were added. An organic layer was extracted three times with chloroform (100 mL) and dried over anhydrous MgSO₄. A crude product was concentrated by using a rotary evaporator and then purified by silica gel column chromatography using acetone:n-hexane (1:3 v/v) as an eluent. A final product was obtained as a dark red solid (2.33 g, 9.9%) and was used without further purification for removing isomers.

¹H-NMR (400 MHz, CDCl₃): δ=8.81 (dd, J=3.9, 1.2 Hz, 1H), 8.38 (dd, J=3.8, 1.1 Hz, 1H), 7.68 (dd, J=5.0, 1.1 Hz, 1H), 7.61 (dd, J=5.0, 1.1 Hz, 1H), 7.31-7.20 (m, 2H), 4.55 (d, J=5.4 Hz, 2H), 4.03 (d, J=7.7 Hz, 1H), 1.89 (s, 1H), 1.29-1.21 (m, 40H), 0.89-0.86 (t, 9H). ¹³C NMR (101 MHz, CDCl₃) δ=161.67, 140.83, 136.27, 135.33, 132.13, 130.81, 130.67, 129.81, 129.13, 129.01, 128.39, 108.41, 77.33, 77.21, 77.01, 76.69, 46.25, 37.75, 31.93, 31.48, 31.17, 30.01, 29.99, 29.71, 29.68, 29.64, 29.55, 29.37, 26.88, 26.21, 22.70, 14.13, 0.00. HR-MS (MALDI-TOF): calcd. for C₄₂H₆₄N₂O₂S₂ (M⁺) 692.441; Found: 693.595.

### 3) Synthesis of Intermediate 5

DPP-DH-H (4) (1.16 g, 1.67 mmol) and K₂CO₃ (460.7 mg, 3.33 mmol) were added to anhydrous DMF (55.6 mL). A solution was stirred at a temperature of 120° C. for 1 hour. Thereafter, n-C₁₄H₂₉Br (693.2 mg, 2.50 mmol) was added to a reaction solution and stirred at a temperature of 120° C. for 16 hours. A reaction mixture was cooled to room temperature, a solvent was removed, and then deionized water (50 mL) and chloroform (50 mL) were added. An organic layer was extracted three times with chloroform (50 mL) and dried over anhydrous MgSO₄. A crude product was concentrated by using a rotary evaporator and then purified by silica gel column chromatography using chloroform:n-hexane (2:1 v/v) as an eluent. A final product was obtained as a dark red solid (1.16 g, 78%).

¹H-NMR (400 MHz, CDCl₃): δ=8.95 (dd, J=3.9, 1.2 Hz, 1H), 8.84 (dd, J=3.9, 1.2 Hz, 1H), 7.62 (td, J=5.1, 1.1 Hz, 2H), 7.31-7.25 (m, 2H), 4.09-4.04 (m, 2H), 4.01 (d, J=7.7 Hz, 2H), 1.93-1.84 (m, 1H), 1.79-1.70 (m, 2H), 1.46-1.14 (m, 70H), 0.88 (t, J=6.8 Hz, 9H). ¹³C NMR (101 MHz, CDCl₃) δ=161.74, 161.37, 140.36, 140.08, 135.37, 135.06, 130.60, 130.49, 129.84, 129.78, 128.62, 128.36, 108.08, 107.56, 77.33, 77.22, 77.01, 76.69, 46.21, 42.26, 37.74, 31.94, 31.17, 30.01, 29.97, 29.71, 29.70, 29.68, 29.66, 29.64, 29.58, 29.56, 29.37, 29.26, 26.90, 26.21, 22.70, 14.13, 0.00. HR-MS (MALDI-TOF): calcd. for C₅₆H₉₂N₂O₂S₂ (M⁺) 888.660; Found: 889.839.

### 4) Synthesis of Intermediate 6

DPP-DH-T (5) (1.16 g, 1.30 mmol) was dissolved in anhydrous DCM (129 mL). A solution was cooled to 0° C. A bromine solution (1 mL, 2.599 M) diluted with DCM was slowly added dropwise to a reaction solution. Afterwards, a reaction mixture was stirred at a temperature of 0° C. for 30 minutes, and the reaction was stopped by using a saturated Na₂S₂O₃ aqueous solution. An organic layer was extracted three times with DCM (100 mL) and dried over anhydrous MgSO₄. After an organic solvent was removed by using a rotary evaporator, a crude product was purified by silica gel column chromatography using chloroform:n-hexane (2:3 v/v) as an eluent. Thereafter, additional recrystallization was performed by using a MeOH/chloroform mixed solvent to obtain a final target compound as a dark red solid (821 mg, 60%).

¹H-NMR (400 MHz, CDCl₃): δ=8.70 (dd, J=4.3, 1.7 Hz, 1H), 8.60 (dd, J=4.2, 1.7 Hz, 1H), 7.23 (ddd, J=7.6, 4.2, 1.7 Hz, 2H), 4.01-3.95 (m, 2H), 3.92 (d, J=7.7 Hz, 2H), 1.86 (s, 1H), 1.72 (m, 2H), 1.23 (m, 70H), 0.93-0.85 (m, 9H). ¹³C NMR (101 MHz, CDCl₃) δ=161.40, 161.03, 139.31, 139.08, 135.49, 135.17, 131.67, 131.41, 131.18, 131.09, 119.11, 118.97, 108.16, 107.67, 77.33, 77.21, 77.01, 76.69, 46.33, 42.32, 37.77, 31.94, 31.16, 29.97, 29.72, 29.70, 29.68, 29.64, 29.56, 29.50, 29.38, 29.20, 26.84, 26.18, 22.70, 14.13, 0.00. HR-MS (MALDI-TOF): calcd. for C₅₆H₉₀Br₂N₂O₂S₂ (M⁺) 1047.480, Found: 1047.699.

### 5) Preparation of PDPP-1

DPP-DH-T-Br₂ (6) (400 mg, 0.38 mmol), 2,5-bis(trimethylstannyl)thiophene (174.40 mg, 0.3819 mmol), a tris(dibenzylideneacetone)dipalladium(0)-chloroform adduct (7.9 mg, 7.64×10−3 mmol), and tri(o-tolyl)phosphine (9.3 mg, 3.05×10⁻² mmol) were degassed and dissolved in anhydrous toluene (8.49 mL). A solution was stirred at a temperature of 60° C. and gradually heated to 110° C. at a rate of 1° C./min. After 20 min, 2-bromothiophene (0.1 mL) was added and additionally stirred for 1 hour. After a reaction mixture was cooled to room temperature, in order to remove a catalyst, diethylammonium diethyldithiocarbamate (339.8 mg, 1.53 mmol) dissolved in chloroform (80 mL) and deionized water (100 mL) was stirred at a temperature of 50° C. for 24 hours under a nitrogen atmosphere. A reaction solution was extracted three times with chloroform (60 mL) and cleaned with brine (60 mL) and deionized water (60 mL).

A crude product dissolved in chloroform (10 mL) was precipitated into methanol (400 mL). A collected polymer was additionally purified through Soxhlet extraction by sequentially using methanol, acetone, n-hexane, cyclohexane, DCM, and chloroform. A chloroform fraction was precipitated again into methanol and filtered. Finally, a PDPP polymer was obtained in the form of a blue solid in a yield of 95% (371 mg).

¹H-NMR (400 MHz, CDCl₃): δ=9.20-8.75 (br), 7.10-6.3 (br), 8.10-7.85 (br, 2H), 7.50-7.35 (br, 2H), 1.48-1.09 (br), 1.02-0.80 (br). GPC: Mₙ=81,000 Da, M_{w}=197,000 Da, PDI=2.43

### (2) Preparation of Compound PNDI-1

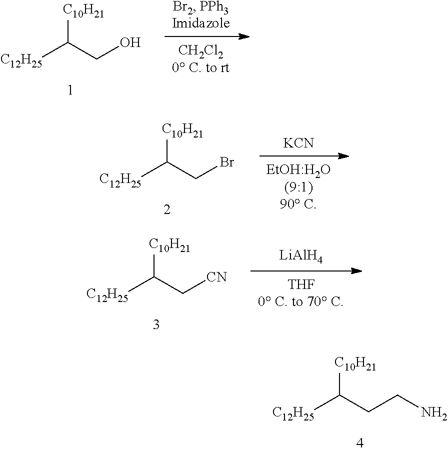

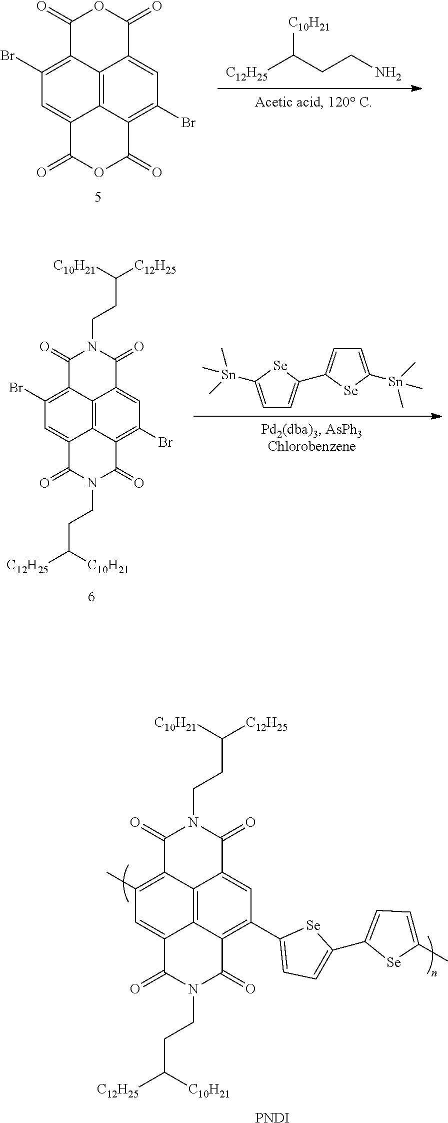

NDI3DP-Br₂ (6) (600 mg, 0.53 mmol), 5,5′-bis(trimethylstannyl)-2,2′-biselenophene (312.28 mg, 0.53 mmol), tris(dibenzylideneacetone)dipalladium(0) (14.65 mg, 3 mol %), and triphenylarsine (19.60 mg, 12 mol %) were degassed under a nitrogen atmosphere and dissolved in anhydrous chlorobenzene (8.02 mL). A mixture was immediately heated to 60° C., and then a reaction solution was gradually heated to 110° C. at a rate of 1° C. every 2 minutes. After 6 hours, 2-bromothiophene (0.1 mL) was added to a solution and stirred for 1 hour. Afterwards, a reaction mixture was cooled to room temperature, and diethylammonium diethyldithiocarbamate (413.70 mg, 1.86 mmol) dissolved in chloroform (80 mL) and deionized water (70 mL) was added under a nitrogen atmosphere to remove a catalyst and was continuously stirred at a temperature of 50° C. for 24 hours.

A reaction solution was extracted three times with chloroform (80 mL) and then cleaned with each of brine (50 mL) and deionized water (50 mL). A crude product was dissolved in chloroform (8 mL) and precipitated into methanol (300 mL). A collected polymer was additionally purified through Soxhlet extraction by sequentially using methanol, acetone, n-hexane, cyclohexane, DCM, and chloroform. A chloroform fraction was precipitated again into methanol and filtered. Finally, polymer PNDI-1 (602 mg, 92%) was obtained in the form of a blue solid.

¹H-NMR (400 MHz, CDCl₃): δ=8.80-7.80 (br), 1.40-1.1 (br), 0.97-0.79 (br). GPC: Mₙ=95,000 Da, M_{w}=190,000 Da, PDI=2.00.

### Example 1: Preparation of Pattern Film Using PDPP-1 and Crosslinkinq Compound 2 (Diazo-6Bx) (0.88 Mol %)

A highly p-doped Si wafer with a 100 nm-thick SiO₂ layer was sequentially cleaned with acetone, isopropyl alcohol, and water for 10 min each by using an ultrasonic cleaner. A cleaned Si wafer was surface-treated by using octadecyltrichlorosilane (ODTS). 5 mg of PDPP-1 (organic semiconductor material), 0.88 mol % of a diazo-6Bx crosslinking compound, and 1 mL of chloroform were added and stirred at room temperature to prepare a composition for a crosslinking compound. In a glove box under an argon environment, the Si wafer was spin-coated with the composition for a crosslinking compound to prepare a thin film. A photomask having a pattern was disposed on the thin film and exposed to ultraviolet light to crosslink the composition for a crosslinking compound. Afterwards, a non-crosslinked portion was cleaned with chloroform in a spin state by using a spin coater. The wafer was stored in a glove box under an argon environment for 12 hours to remove any remaining solvent, thereby preparing a pattern film that includes PDPP-1 including crosslinking compound 2.

### Comparative Example 1-1: Preparation of Pattern Film Using PDPP-1 and Compound 4Bx (0.88 Mol %)

A pattern film was prepared in the same manner as in Example 1, except that compound 4Bx was used instead of a diazo-6Bx crosslinking compound.

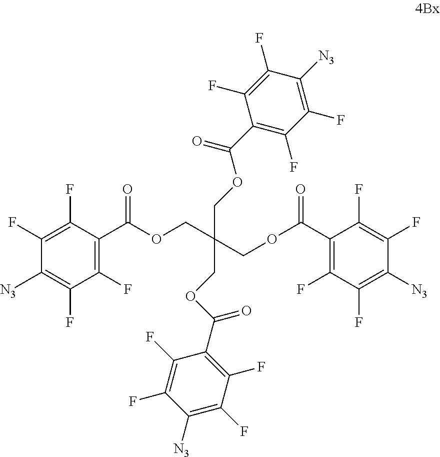 [SMILES: [N-]=[N+]=Nc1c(F)c(F)c(C(=O)OCC(COC(=O)c2c(F)c(F)c(N=[N+]=[N-])c(F)c2F)(COC(=O)c2c(F)c(F)c(N=[N+]=[N-])c(F)c2F)COC(=O)c2c(F)c(F)c(N=[N+]=[N-])c(F)c2F)c(F)c1F]

### Comparative Example 1-2: Preparation of Pattern Film Using PDPP-1 and Compound 6Bx (0.88 Mol %)

A pattern film was prepared in the same manner as in Example 1, except that compound 6Bx was used instead of a diazo-6Bx crosslinking compound.

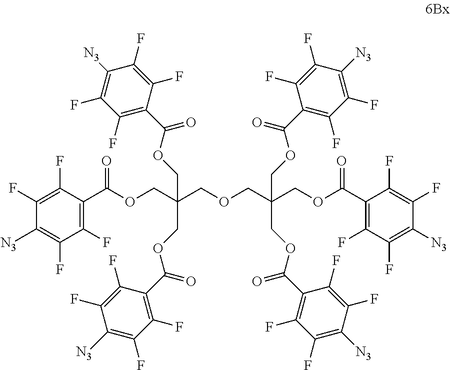 [SMILES: [N-]=[N+]=Nc1c(F)c(F)c(C(=O)OCC(COCC(COC(=O)c2c(F)c(F)c(N=[N+]=[N-])c(F)c2F)(COC(=O)c2c(F)c(F)c(N=[N+]=[N-])c(F)c2F)COC(=O)c2c(F)c(F)c(N=[N+]=[N-])c(F)c2F)(COC(=O)c2c(F)c(F)c(N=[N+]=[N-])c(F)c2F)COC(=O)c2c(F)c(F)c(N=[N+]=[N-])c(F)c2F)c(F)c1F]

### Comparative Example 1-3: Preparation of Pattern Film Using PDPP-1 and Compound pH-6Bx (0.88 Mol %)

A pattern film was prepared in the same manner as in Example 1, except that a compound Ph-6Bx was used instead of a diazo-6Bx crosslinking compound.

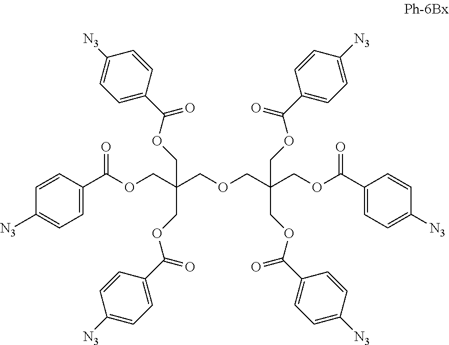 [SMILES: [N-]=[N+]=Nc1ccc(C(=O)OCC(COCC(COC(=O)c2ccc(N=[N+]=[N-])cc2)(COC(=O)c2ccc(N=[N+]=[N-])cc2)COC(=O)c2ccc(N=[N+]=[N-])cc2)(COC(=O)c2ccc(N=[N+]=[N-])cc2)COC(=O)c2ccc(N=[N+]=[N-])cc2)cc1]

### Evaluation Example 1: Confirmation of Pattern Height Profile of Pattern Film

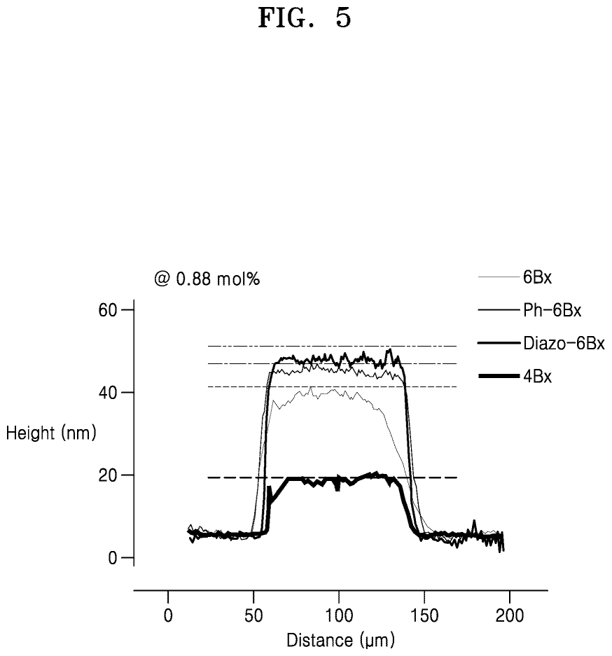

Height profiles of patterns of Example 1 and Comparative Examples 1-1 to 1-3 were confirmed. Results thereof are shown in FIG. 5. A profile of the pattern was measured by using a DektakXT surface profiler manufactured by Bruker.

Referring to FIG. 5, it was confirmed that a pattern height of the pattern film formed according to Example 1 was most ideally formed, resulting in high pattern fidelity and high pattern height retention.

### Example 2: Preparation of Pattern Film Using PDPP-1 and Crosslinkinq Compound 2 (Diazo-6Bx) (2.64 Mol %)

A highly p-doped Si wafer with a 100 nm thick SiO₂ layer was sequentially cleaned with acetone, isopropyl alcohol, and water for 10 min each by using an ultrasonic cleaner. A cleaned Si wafer was surface-treated by using ODTS. 5 mg of PDPP-1 (organic semiconductor material), 2.64 mol % of a PDPP-1 crosslinking compound, and 1 mL of chloroform were added and stirred at room temperature to prepare a composition for a crosslinking compound. In a glove box under an argon environment, the Si wafer was spin-coated with the composition for a crosslinking compound to prepare a thin film. A photomask having a pattern was disposed on the thin film and exposed to ultraviolet light to crosslink the composition for a crosslinking compound. Afterwards, a non-crosslinked portion was cleaned with chloroform in a spin state by using a spin coater. The wafer was stored in a glove box under an argon environment for 12 hours to remove any remaining solvent, thereby preparing a pattern film that includes PDPP-1 including crosslinking compound 2.

### Comparative Example 2-1: Preparation of Pattern Film Using PDPP-1 and Compound 6Bx (2.64 Mol %)

A pattern film was prepared in the same manner as in Example 2, except that compound 6Bx was used instead of a diazo-6Bx crosslinking compound.

### Comparative Example 2-2: Preparation of Pattern Film Using PDPP-1 and Compound pH-6Bx (2.64 Mol %)

A pattern film was prepared in the same manner as in Example 2, except that compound Ph-6Bx was used instead of a diazo-6Bx crosslinking compound.

### Evaluation Example 2: Confirmation of Pattern Height, Width Profile, and Taper Angle of Pattern Film

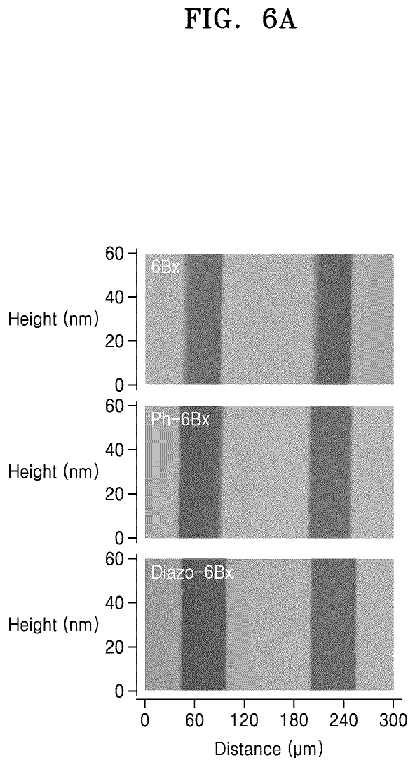

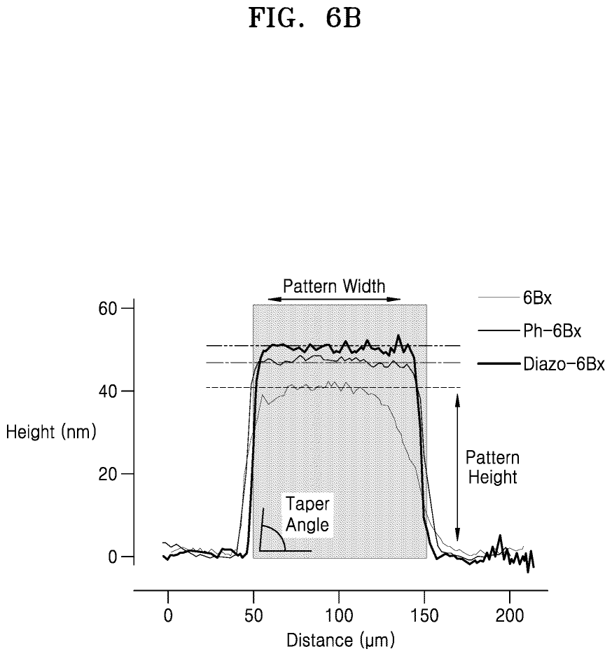

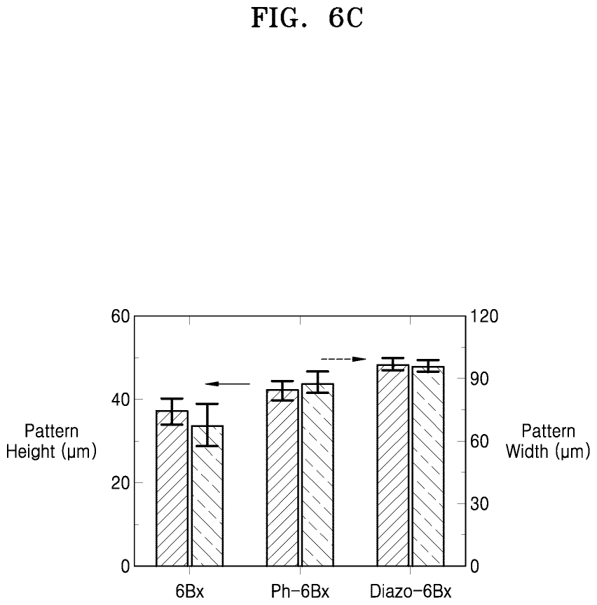

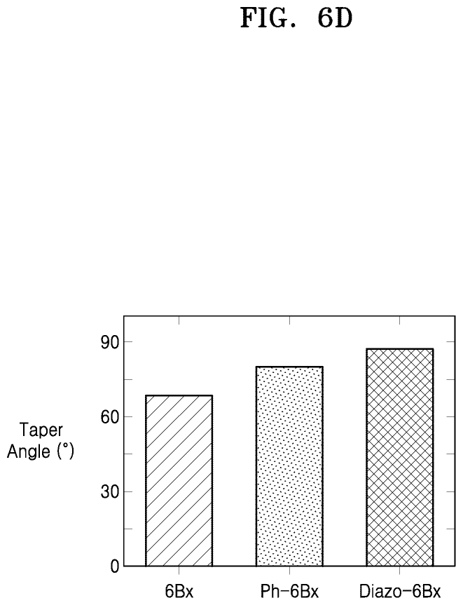

Height profiles, width profiles, and taper angles of patterns of Example 2 and Comparative Examples 2-1 to 2-2 were confirmed. Results thereof are shown in FIGS. 6A to 6D. A profile of the pattern was measured by using a DektakXT surface profiler manufactured by Bruker.

Referring to FIGS. 6A to 6D, it was confirmed that the pattern height, width, and taper angle of the pattern film formed according to Example 2 were most ideally formed, resulting in high pattern fidelity and high pattern height retention.

### Evaluation Example 3: Confirmation of Pattern Height Retention According to Crosslinking Compound Fraction

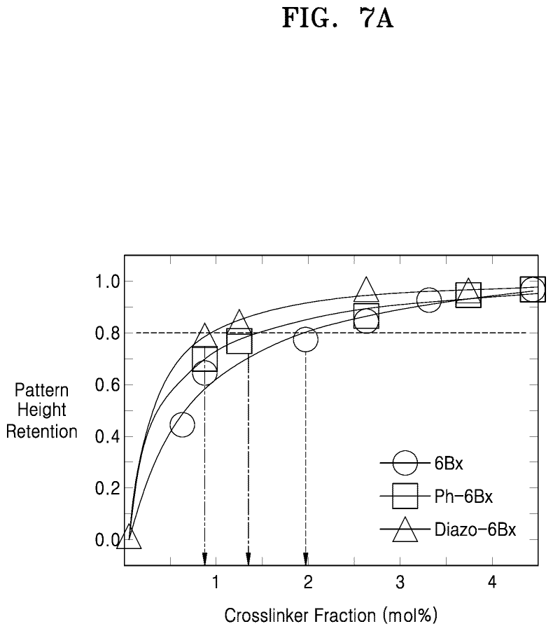

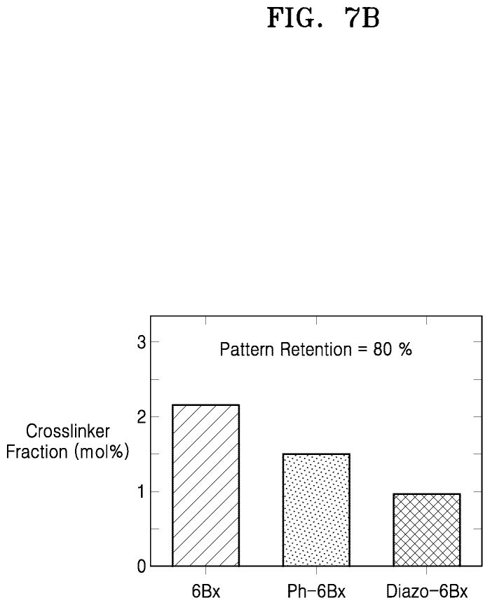

For diazo-6Bx (crosslinking compound 2), compound 6Bx, and compound Ph-6Bx, pattern height retention according to crosslinking compound fraction was confirmed. Results thereof are shown in FIG. 7A. In addition, a crosslinking compound fraction required to achieve a pattern height retention of 0.8 was measured. Results thereof are shown in FIG. 7B.

Referring to FIGS. 7A and 7B, it was confirmed that the pattern height retention of a pattern film formed by using diazo-6Bx (crosslinking compound 2) was the highest, and it was also confirmed that a crosslinking compound fraction required to achieve a pattern height retention of 0.8 was the lowest for diazo-6Bx (crosslinking compound 2).

Therefore, it was confirmed that a pattern or pattern film using a crosslinking composition including crosslinking compound 2 had the best pattern fidelity, thereby indicating that the crosslinking efficiency of crosslinking compound 2 was the highest.

### Evaluation Example 4: Confirmation of Miscibility and Compatibility with Compounds PDPP-1 and PNDI-1 According to Crosslinking Compounds (Confirmation of Hansen Solubility Parameters)

TABLE 1

| | δ_{D} | δ_{P} | δ_{H} | | | | | |
|---|---|---|---|---|---|---|---|---|
| | MPa^{0.5} | MPa^{0.5} | MPa^{0.5} | R₀ | R_{a, DPP} | R_{a, NDI} | RED_{DPP} | RED_{NDI} |
| 6Bx | 19.5 | 11.5 | 4.5 | 12.0 | 7.228 | 8.587 | 0.602 | 0.716 |
| Ph-6Bx | 20.0 | 10.6 | 4.5 | 11.8 | 6.828 | 7.951 | 0.581 | 0.676 |
| Diazo-6Bx | 19.9 | 10.1 | 4.5 | 12.0 | 6.297 | 7.420 | 0.525 | 0.618 |

For diazo-6Bx (crosslinking compound 2), compound 6Bx, and compound Ph-6Bx, Hansen solubility parameters were conformed to confirm compatibility with compound PDPP-1 and compound PNDI-1 which were organic semiconductor materials. Results thereof are shown in Table 1. When an RED value is less than 1, and particularly close to 0, very high miscibility and compatibility may be exhibited.

Referring to Table 1, crosslinking compound 2 has the lowest RED value for all organic semiconductor materials and possesses the highest miscibility and compatibility. Thus, crosslinking compound 2 has better interaction with the organic semiconductor material to exhibit high crosslinking efficiency through a uniform distribution throughout the entire uniform thin film, thereby achieving high pattern fidelity. In addition, after crosslinking, the proper overlapping between pi-conjugated backbones of an organic semiconductor may be strengthened so that a charge transport path may be well maintained.

### Device Manufacturing Example 1: Manufacturing of OTFT Using Crosslinking Compound 2 (Diazo-6Bx)

A substrate was sequentially cleaned with acetone, isopropyl alcohol, and water for 10 min each by using an ultrasonic cleaner. Each of a source electrode and a drain electrode (Cr electrode and Au electrode) was deposited on the substrate through thermal evaporation by using a shadow mask. Accordingly, a thickness of the formed Cr electrode is about 3 nm, and a thickness of the formed Au electrode is about 17 nm. A channel length and a width of each of the source electrode and the drain electrode are 100 μm and 800 μm, respectively.

5.0 mg of an organic semiconductor material (PDPP-1), 2.64 mol % (0.145 mg) of a diazo-6Bx crosslinking compound, and 1 mL of chloroform were added and stirred at room temperature to prepare a composition for a crosslinking agent. The substrate on which electrodes were formed was spin-coated with the composition for a crosslinking agent at 1,000 rpm for 30 s to form a thin film. A photomask having a pattern was disposed on the thin film and exposed to ultraviolet light to crosslink the composition for a crosslinking agent. Afterwards, a non-crosslinked portion was cleaned with chloroform in a spin state by using a spin coater. The substrate was stored in a glove box under an argon environment for 4 hours to remove any remaining solvent.

In this case, in the case of a logic electronic device, after a p-type organic semiconductor material patterning process using an organic semiconductor material, a solution, which was obtained by adding and stirring 6.02 mg of an n-type organic semiconductor material (PNDI-1), 3.24 mol % (0.178 mg) of diazo-6Bx, and 1 mL of chloroform, was applied at 1,000 rpm for 30 s through spin coating to prepare a thin film. A photomask was disposed on the thin film, and crosslinking was performed by exposure to ultraviolet light. A non-crosslinked portion was cleaned with chloroform in a spin state by using a spin coater, and the substrate was stored in a glove box under a nitrogen environment for 12 hours to remove any remaining solvent. Afterwards, a solution in which 70 mg of PMMA and 17.5 mg of a diazo-6Bx crosslinking compound were dissolved in 1 mL of n-butyl acetate was applied at 1,000 rpm for 60 s through spin coating to prepare a thin film, and crosslinking was performed by exposure to ultraviolet light. After drying in a vacuum oven at a temperature of 80° C. for 12 hours, a thickness of a polymer insulating layer was 400 nm. Au used as a gate electrode was deposited on the polymer insulating layer to have a thickness of 40 nm, thereby manufacturing an OTFT.

### Comparative Device Manufacturing Example 1: Manufacturing of OTFT Using Compound 6Bx

A pattern film was prepared in the same manner as in Device Manufacturing Example 1, except that compound 6Bx was used instead of a diazo-6Bx crosslinking compound.

### Comparative Device Manufacturing Example 2: Manufacturing of OTFT Using Compound pH-6Bx

A pattern film was prepared in the same manner as in Device Manufacturing Example 1, except that compound Ph-6Bx was used instead of a diazo-6Bx crosslinking compound.

### Evaluation Example 5: Confirmation of Operational Stability and Environmental Stability of OTFT

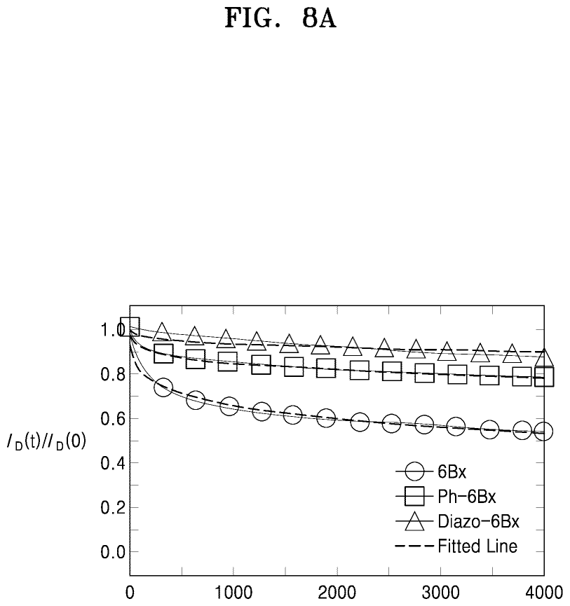

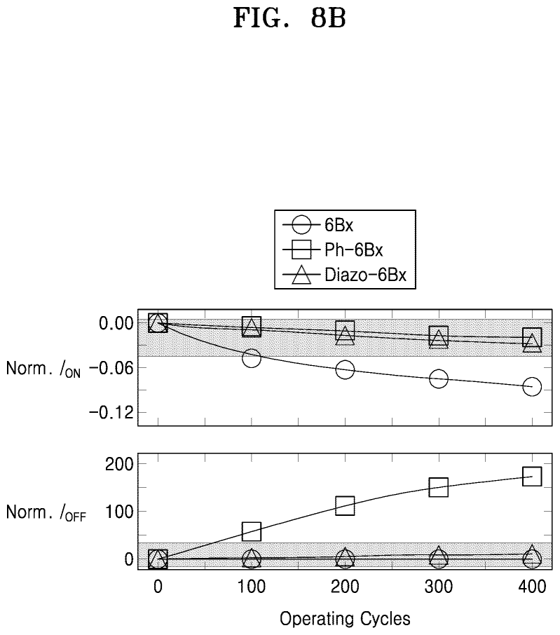

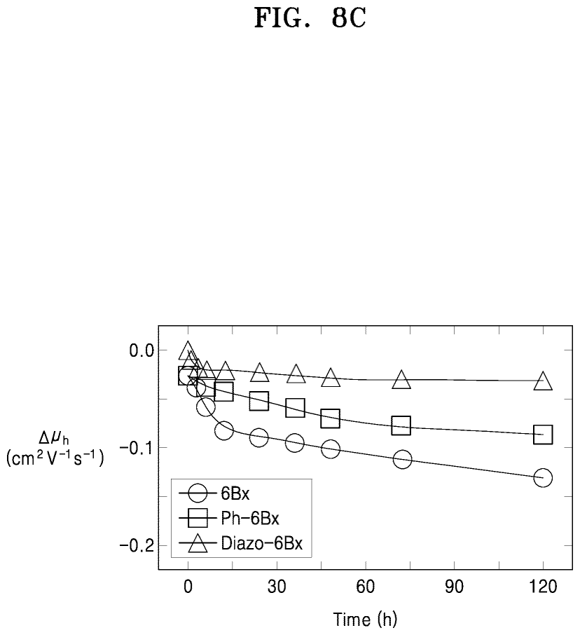

The operational stability and environmental stability of the OTFTs according to Device Manufacturing Example 1 and Comparative Device Manufacturing Examples 1 and 2 were confirmed. Results thereof are shown in FIGS. 8A to 8C.

Referring to FIG. 8A, a bias-stress effect of PDPP-channel OTFTs is shown. A bias of V_{G} of −60 V and a bias of V_{D} of −20 V were continuously applied to a p-type PDPP OTFT for 4,000 s. While the OTFTs of Comparative Device Manufacturing Examples 1 and 2 showed an abrupt decrease in current, the OTFT according to Device Manufacturing Example 1 showed a gradual decrease in current during an entire period of 4,000 s, thereby confirming high operational stability.

Referring to FIG. 8B, as a result in which measurement was performed for 3,000 s or more while a square pulse voltage V_{G} of −50 V to +10 V was applied, and V_{D} was maintained at −10 V, it was confirmed that the OTFT according to Device Manufacturing Example 1 exhibited a minimal on/off current change and thus had high operational stability.

Referring to FIG. 8C, a change of a transfer curve was tracked for 120 hours under room temperature and atmospheric conditions to evaluate environmental stability. In the case of Device Manufacturing Example 1, it was confirmed that, since a μₕ change amount was the lowest, the OTFT had the highest environmental stability.

### Evaluation Example 6: Confirmation of Carrier Mobility Characteristics of OTFT

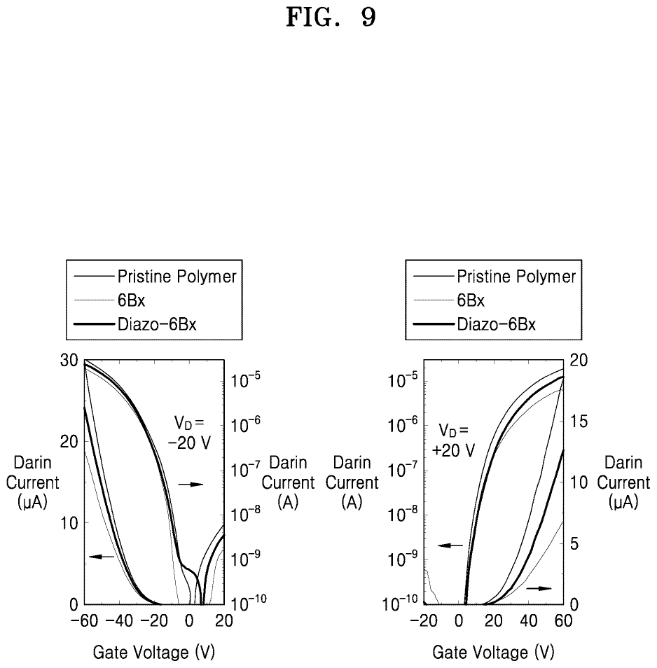

Carrier mobility of each of the OTFTs according to Device Manufacturing Example 1 and Comparative Device Manufacturing Example 1 was calculated from a transfer curve. Results thereof are shown in FIG. 9 and Table 2.

The carrier mobility of the OTFT was calculated by using the following equation:

$${I}_{D} = ( μ \cdot C ) \cdot {( {V}_{G} - {V}_{TH} )}^{2} \cdot ( 2 {WL}^{- 1} )$$

Here, I_{D} denotes a drain current, V_{G} denotes a gate voltage, C denotes capacitance per unit area of a PMMA gate insulating film, V_{TH} denotes a threshold voltage, and W and L denote a width and a length of a channel, respectively.

TABLE 2

| | Comparative Device | Device Manufacturing |
|---|---|---|
| | Manufacturing Example 1 | Example 1 |
| | Carrier mobility | Carrier mobility |
| | 6Bx | Diazo-6Bx |
| Polymer | 2.64 mol % | 2.64 mol % |
| PDPP-1 | 0.92 ± 0.02 cm²V⁻¹s⁻¹ | 1.01 ± 0.04 cm²V⁻¹s⁻¹ |
| | 6Bx | Diazo-6Bx |
| Polymer | 3.24 mol % | 3.24 mol % |
| PNDI-1 | 0.78 ± 0.03 cm²V⁻¹s⁻¹ | 0.92 ± 0.04 cm²V⁻¹s⁻¹ |

In this case, FIG. 9 shows transfer curves of OTFTs based on PDPP-1 and PNDI-1, respectively. It was confirmed that the OTFTs respectively exhibited typical p-channel operating characteristics and n-channel operating characteristics.

In addition, referring to FIG. 9 and Table 2, it was confirmed that the carrier mobility of the OTFT according to Device Manufacturing Example 1 was superior to that of the OTFT according to Comparative Device Manufacturing Example 1, and thus it was confirmed that a switching speed of the OTFT according to the disclosure was excellent.

According to the disclosure, a manufacturing process of forming a pattern and/or manufacturing an electronic device may be simplified, and damage to components of an ecteronic device may be simultaneously prevented.

In addition, since a crosslinking compound according to the disclosure has excellent compatibility and miscibility with organic semiconductor materials (for example, host materials), general-purpose polymer materials, quantum dot materials, nanocrystal materials, and other materials, a pattern formed by using a crosslinking composition according to the disclosure may have high fidelity and achieve excellent crosslinking efficiency.

In addition, since a pattern film and an electronic device formed through a pattern formation method using the crosslinking composition have superior mobility as compared to electronic devices using an improved crosslinking composition according to a related art, high-quality electronic devices may be manufactured by using the crosslinking composition according to the disclosure.

While the disclosure has been described with the above embodiments, this is merely illustrative, and those skilled in the art will understand that various modifications and other equivalent embodiments are possible therefrom. Therefore, the true technical scope of the disclosure should be defined by the technical spirit of the appended claims

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

1. A crosslinking composition comprising:
a crosslinking compound comprising a diazo group; and
an organic semiconductor material, a polymer material, a metal material, an insulating material, or any combination thereof.

2. The crosslinking composition of claim 1, wherein the organic semiconductor material comprises a conjugated polymer, a conjugated small molecule, a poly(diketopyrrolopyrrole) (PDPP)-based organic semiconductor material, a poly(naphthalene diimide) (PNDI)-based organic semiconductor material, or any combination thereof.

3. The crosslinking composition of claim 1, wherein the polymer material comprises a C—H bond.

4. The crosslinking composition of claim 1, wherein the crosslinking compound is represented by Formula 1 below: 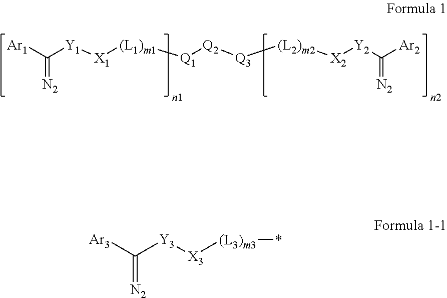
wherein, in Formulas 1 and 1-1,
L₁ to L₃ are each independently a single bond or a C₁-C₃₀ alkylene group unsubstituted or substituted with at least one R₁,
m1 to m3 are each independently 0, 1, 2, 3, 4, 5, or 6,
Ar₁ to Ar₃ are each independently a C₅-C₆₀ carbocyclic group unsubstituted or substituted with at least one R₁ or a C₁-C₆₀ heterocyclic group unsubstituted or substituted with at least one R₁,
n1 and n2 are each independently 0, 1, 2, 3, 4, 5, 6, 7, 8, 9, or 10,
the sum of n1 and n2 is greater than or equal to 2,
Q₁ to Q₃ are each independently a single bond, O, S, C, C(R₂), C(R₂)(R₃), a C₁-C₃₀ alkylene group unsubstituted or substituted with at least one R₁, or any combination thereof,
X₁ to X₃ are each independently O, S, Se, N(R₄), or C(R₄)(R₅),
Y₁ to Y₃ are each independently *—C(═O)—*′, *—C(═N(R₆))—*′, *—O—*′, *—S—*′, *—Se—*′, *—N(R₆)—*′, or *—C(R₆)(R₇)—*′,
R₁ to R₇ are each independently a group represented by Formula 1-1, hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, a C₁-C₃₀ alkylthio group, a C₅-C₆₀ carbocyclic group, a C₁-C₆₀ heterocyclic group, or —Si(Q₁₁)(Q₁₂)(Q₁₃),
Q₁₁ to Q₁₃ are each independently hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group, and
* and *′ is a binding site with an adjacent atom.

5. The crosslinking composition of claim 4, wherein the crosslinking compound is represented by Formula 2 below: 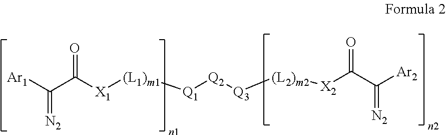
wherein, in Formula 2, L₁, L₂, m1, m2, n1, n2, X₁, X₂, Ar₁, Ar₂, and Q₁ to Q₃ are as defined in claim 4.

6. The crosslinking composition of claim 4, wherein the crosslinking compound is represented by Formula 3 below: 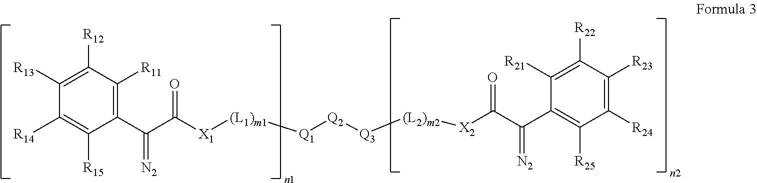
wherein, in Formula 3,
L₁, L₂, m1, m2, n1, n2, X₁, X₂, and Q₁ to Q₃ are as defined in claim 4, and
R₁₁ to R₁₅ and R₂₁ to R₂₅ are each independently defined as for R₁ of claim 4.

7. The crosslinking composition of claim 1, wherein the crosslinking compound is at least one selected from Compounds 1 to 7 below:

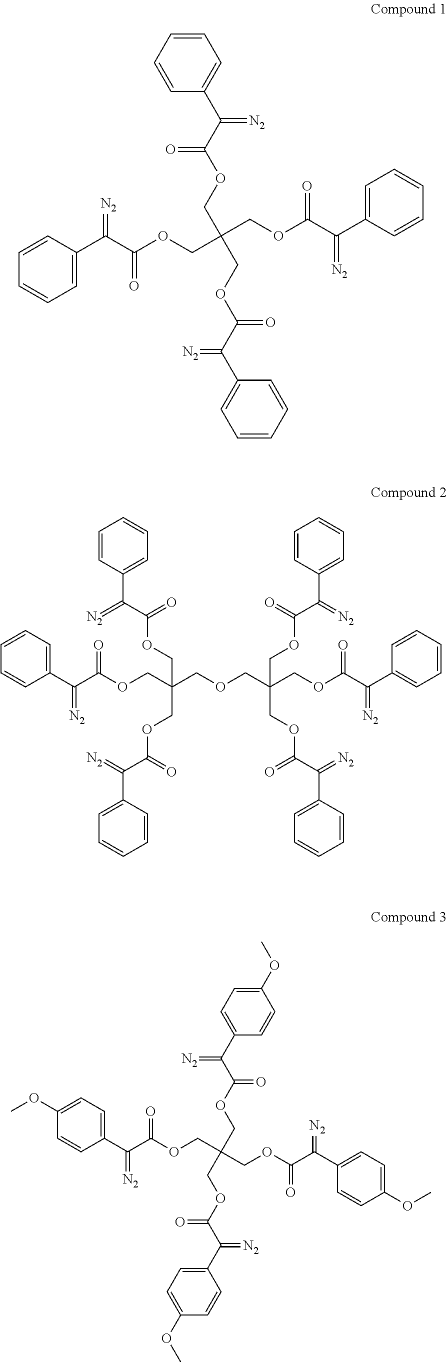

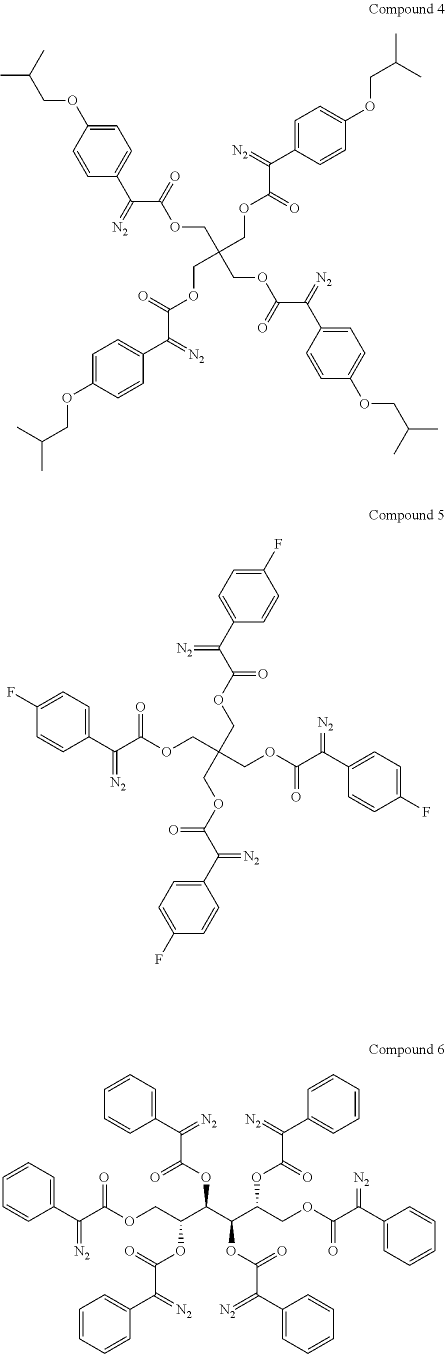

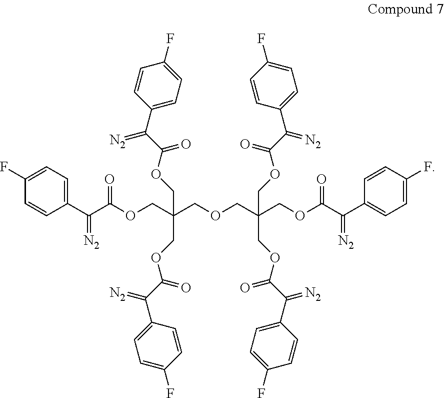

8. The crosslinking composition of claim 1, wherein the crosslinking compound is a low-temperature-activated crosslinking compound that is thermally activated at a temperature of about 80° C. to about 130° C. to generate an intermediate.

9. The crosslinking composition of claim 1, wherein the organic semiconductor material is at least one selected from compounds PDPP-1 and PNDI-1:

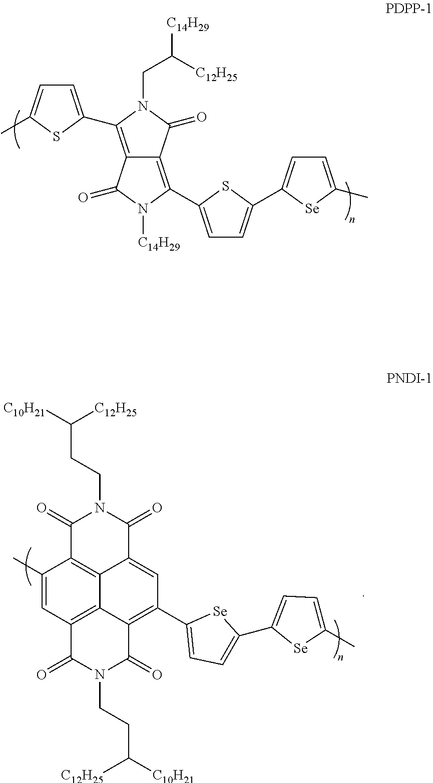

10. A pattern formation method comprising:
forming a lower film by applying a crosslinking composition onto a substrate; and
patterning the lower film,
wherein the crosslinking composition comprises:
a crosslinking compound comprising a diazo group; and
an organic semiconductor material, a polymer material, a metal material, an insulating material, or any combination thereof.

11. The pattern formation method of claim 10, wherein the patterning of the lower film comprises:
arranging a photomask on the lower film;
irradiating light onto the photomask; and
removing a portion of the lower film.

12. The pattern formation method of claim 10, wherein the organic semiconductor material comprises a conjugated polymer, a conjugated small molecule, a poly(diketopyrrolopyrrole) (PDPP)-based organic semiconductor material, a poly(naphthalene diimide) (PNDI)-based organic semiconductor material, or any combination thereof.

13. The pattern formation method of claim 10, wherein the crosslinking compound is represented by Formula 1 below: 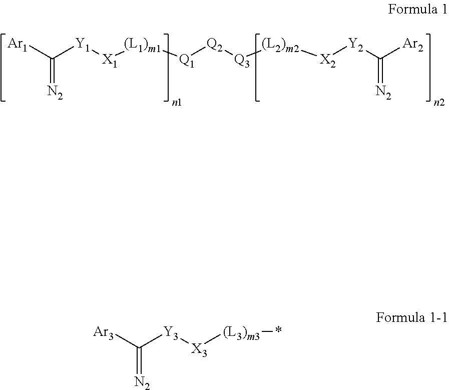
wherein, in Formulas 1 and 1-1,
L₁ to L₃ are each independently a single bond or a C₁-C₃₀ alkylene group unsubstituted or substituted with at least one R₁,
m1 to m3 are each independently 0, 1, 2, 3, 4, 5, or 6,
Ar₁ to Ar₃ are each independently a C₅-C₆₀ carbocyclic group unsubstituted or substituted with at least one R₁ or a C₁-C₆₀ heterocyclic group unsubstituted or substituted with at least one R₁,
n1 and n2 are each independently 0, 1, 2, 3, 4, 5, 6, 7, 8, 9, or 10,
the sum of n1 and n2 is greater than or equal to 2,
Q₁ to Q₃ are each independently a single bond, O, S, C, C(R₂), C(R₂)(R₃), a C₁-C₃₀ alkylene group unsubstituted or substituted with at least one R₁, or any combination thereof,
X₁ to X₃ are each independently O, S, Se, N(R₄), or C(R₄)(R₅),
Y₁ to Y₃ are each independently *—C(═O)—*′, *—C(═N(R₆))—*′, *—O—*′, *—S—*′, *—Se—*′, *—N(R₆)—*′, or *—C(R₆)(R₇)—*′,
R₁ to R₇ are each independently a group represented by Formula 1-1, hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, a C₁-C₃₀ alkylthio group, a C₅-C₆₀ carbocyclic group, a C₁-C₆₀ heterocyclic group, or —Si(Q₁₁)(Q₁₂)(Q₁₃),
Q₁₁ to Q₁₃ are each independently hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group, and
* and *′ is a binding site with an adjacent atom.

14. The pattern formation method of claim 13, wherein the crosslinking compound is represented by Formula 2 below: 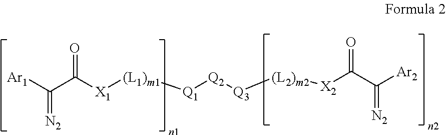
wherein, in Formula 2, L₁, L₂, m1, m2, n1, n2, X₁, X₂, Ar₁, Ar₂, and Q₁ to Q₃ are as defined in claim 13.

15. The pattern formation method of claim 10, wherein the crosslinking compound is a low-temperature-activated crosslinking compound that is thermally activated at a temperature of about 80° C. to about 130° C. to generate an intermediate.

16. The pattern formation method of claim 10, wherein the crosslinking compound is at least one selected from Compounds 1 to 7 below:

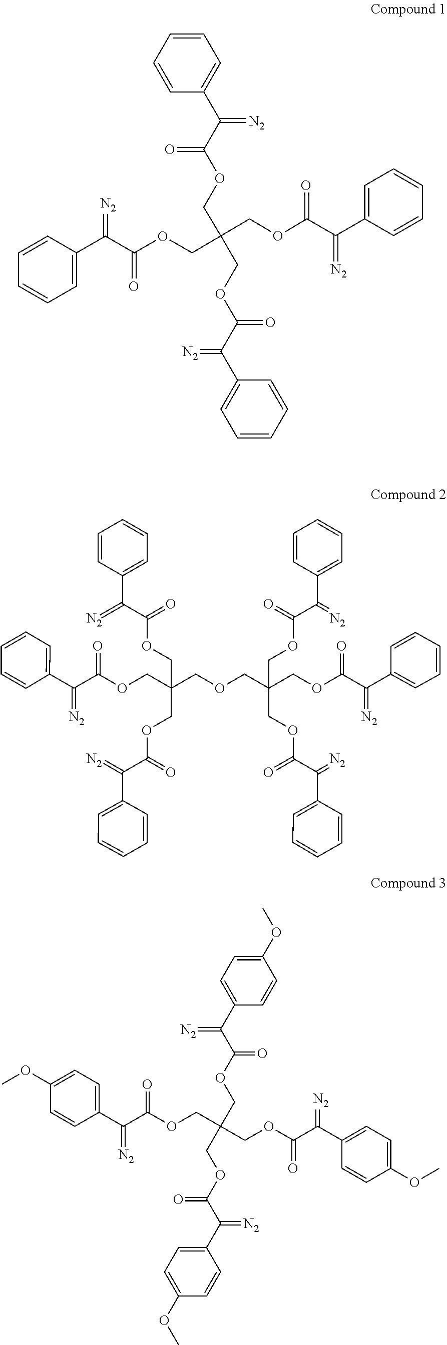

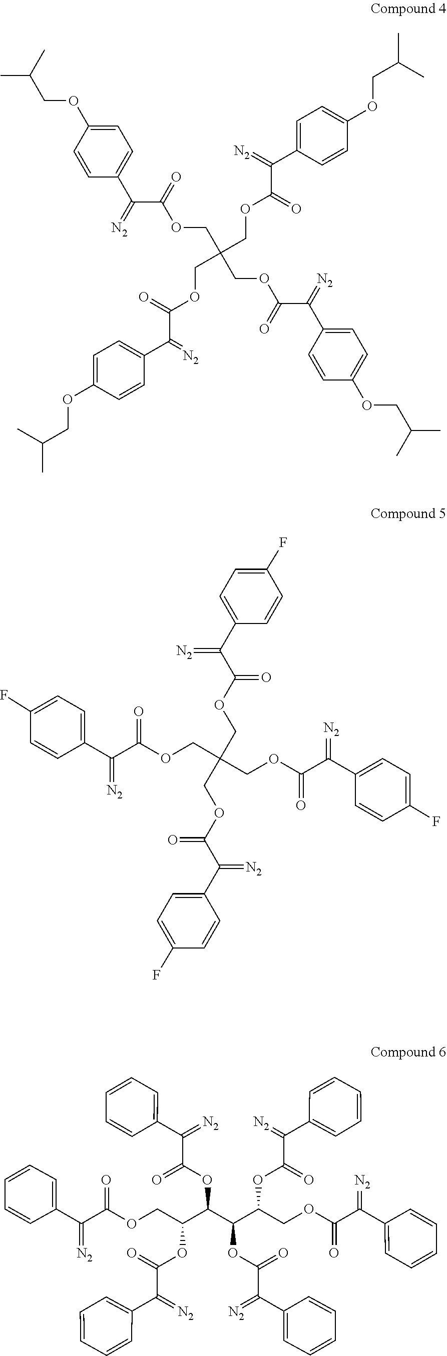

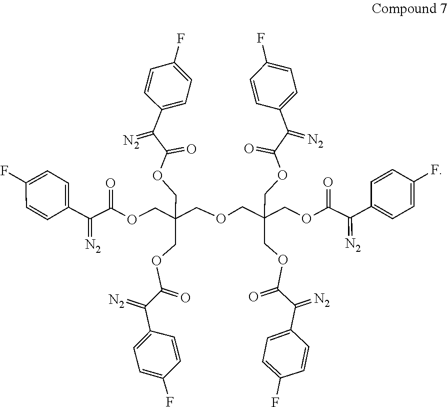

17. A pattern film formed by using a crosslinking composition, the pattern film comprising:
a crosslinking compound comprising a diazo group, and
an organic semiconductor material, a polymer material, a metal material, an insulating material, or any combination thereof.

18. An electronic device including a pattern formed by using a crosslinking composition, the electronic device comprising:
a crosslinking compound comprising a diazo group; and
an organic semiconductor material, a polymer material, a metal material, an insulating material, or any combination thereof.

19. The electronic device of claim 18, wherein the electronic device is an organic thin-film transistor, a logic electronic device, an organic light-emitting diode (OLED), a quantum dot light-emitting diode (QD-LED), an electrochromic (EC) device, a solar cell, a photodiode, or an image sensor.

20. A color filter including a pattern formed by using a crosslinking composition, the color filter comprising:
a crosslinking compound comprising a diazo group; and
an organic semiconductor material, a polymer material, a metal material, an insulating material, or any combination thereof.
